(19) United States
(12) Patent Application Publication
(10) Pub. No.: US 2002/0041897 A1
(43) Pub. Date: Apr. 11, 2002

(54) Compositions for sustained release of antineoplastic taxanes, and methods of making and using the same

(76) Inventor: Wenbin Dang, Ellicott City, MD (US)

Correspondence Address: FOLEY, HOAG & ELIOT LLP, ONE POST OFFICE SQUARE, BOSTON, MA, 02109, US

(21) Appl. No.: 09/901,489

(22) Filed: Jul. 9, 2001

Related U.S. Application Data
(60) Provisional application No. 60/216,874, filed on Jul. 7, 2000.
(60) Provisional application No. 60/239,798, filed on Oct. 12, 2000.

(51) Int. Cl. A61K 9/14; A61K 31/337

(52) U.S. Cl. 424/486; 514/449

(57) ABSTRACT

The present invention relates to compositions containing a biocompatible polymer and an antineoplastic taxane, and methods of using and making the same. In certain embodiments, the polymer contains phosphorous-based linkages and may be biodegradable.

### RELATED APPLICATION INFORMATION

This application claims the benefit of priority under 35 U.S.C. Section 119 to Provisional Patent Application No. 60/216,874, filed Jul. 7, 2000, and Provisional Patent Application No. 60/239,798, filed Oct. 12, 2000. These applications are hereby incorporated by reference in their entirety.

### INTRODUCTION

Taxanes, most notably the two currently approved drugs, paclitaxel (TAXOL) and docetaxel (TAXOTERE), are potent antineoplastic agents. Paclitaxel was discovered in the late 1970s in the bark of the rare Pacific yew, and was found to be an effective antineoplastic agent with a mechanism of action different from existing chemotherapeutic agents. Docetaxel is an analog of paclitaxel which may be prepared semisynthetically from a taxane found in more common yews. Taxanes may also be prepared by total synthesis.

In particular, paclitaxel, docetaxel, and other taxanes exert cytotoxic effects by enhancing the polymerization of tubulin, which is an essential protein in the formation of spindle microtubules. The result is the formation of very stable, nonfunctional tubules, which inhibits cell replication and leads to neoplasm cell death. Taxanes are recognized as effective agents in the treatment of many solid tumors which are refractory to other antineoplastic agents.

Paclitaxel is very poorly water soluble (less than 10 μg/mL), and as a result, cannot be practically formulated with water for IV administration. Some formulations of taxol for injection or I.V. infusion have been developed utilizing CREMOPHOR EL (polyoxyethylated castor oil) as the drug carrier because of taxol's aqueous insolubility. For example, taxol supplied by the NCI has been formulated in 50% CREMOPHOR EL and 50% dehydrated alcohol. CREMOPHOR EL, however, is itself toxic and produces, when given in a large volume single dose without taxol, vasodilation, labored breathing, lethargy, hypotension and death in dogs. One of the major difficulties in the administration of paclitaxel is the occurrence of hypersensitivity reactions. These reactions, which include severe skin rashes, hives, flushing, dyspnea, tachycardia, and others, may be attributed at least in part to the high concentrations of alcohol and CREMOPHOR EL used as solvents in the formulation. Standard treatment regimens now call for premedication prior to infusion of the formulated taxol.

Docetaxel is an analog of paclitaxel, and was recently approved for administration to patients with cancer by the United States Food & Drug Administration. Like paclitaxel, docetaxel is poorly soluble in water. The current most preferred solvent used to dissolve docetaxel for pharmaceutical use is polysorbate 80 (Tween 80). Like Polyoxyl 35, polysorbate 80 often causes hypersensitivity reactions in patients. Further, polysorbate cannot be used with PVC delivery apparatus, because of its tendency to leach diethylhexyl phthalate, which is highly toxic.

An improved delivery method that avoids the use of solvents and excipients that promote adverse reactions in patients is needed. Furthermore, a therapeutic regimen using a formulation that reduces the frequency and cost of drug treatments, and possibly reduces the need for conjoint therapies, such as radiotherapy or standard chemotherapy, that have more severe side effects would increase the attractiveness and convenience of taxane therapy.

### SUMMARY OF THE INVENTION

In part, the present invention is directed to a polymer system, such as a biocompatible polymer, comprising paclitaxel, docetaxel or other antineoplastic taxane or an analog thereof, methods for treatment using the subject polymers, and methods of making and using the same.

In certain embodiments, a large percentage of the subject composition may be an antineoplastic taxane. For example, paclitaxel, docetaxel or an analog thereof may comprise 5% to 60% or more of the subject composition, such as at least about 10%, at least about 30%, or at least about 50% of an antineoplastic taxane.

In certain embodiments, administration of the subject polymers results in sustained release of an encapsulated antineoplastic taxane for a period of time and in an amount that is not possible with other modes of administration of such therapeutic agent. In certain embodiments, such administration results in systemic levels of the antineoplastic taxane for a prolonged period.

The subject compositions, and methods of making and using the same, achieve a number of desirable results and features, one or more of which (if any) may be present in any particular embodiment of the present invention: (i) a single dose of a subject composition may achieve the desired therapeutically beneficial response through sustained release of an antineoplastic taxane; (ii) sustained release of an antineoplastic taxane from a biocompatible and possibly biodegradable polymer composition; (iii) novel treatment regimens for treating or preventing cancer and other diseases using the subject compositions for sustained delivery of an antineoplastic agent; (iv) detectable systemic levels of an antineoplastic taxane for a period of time in a patient; (v) high levels of loading (by weight), e.g. greater than 10% and up to 60% or more, of an antineoplastic taxane in biocompatible polymers; (vi) lyophilization or subjection to an appropriate drying technique such as spray drying of the subject compositions and subsequent rehydration; (vii) reduction in hypersensitivity reactions, and (viii) co-encapsulation of therapeutic agents in addition to any antineoplastic taxane in biocompatible and optionally biodegradable polymers.

In one aspect, the subject polymers may be biocompatible, biodegradable or both. In certain embodiments, the subject polymers contain phosphorus linkages, including, for example, phosphate, phosphonate and phosphite. In other embodiments, the monomeric units of the present invention have the structures described in the claims appended below, which are hereby incorporated by reference in their entirety into this Summary. In the subject polymers, and in particular in those embodiments containing a phosphorus linkage, the chemical structure of certain of the monomeric units may be varied to achieve a variety of desirable physical or chemical characteristics, including for example, release profiles or handling characteristics of the resulting polymer composition.

In certain embodiments, other materials may be encapsulated in the subject polymer in addition to paclitaxel, docetaxel or an analog thereof to alter the physical and chemical properties of the resulting polymer, including for example, the release profile of the resulting polymer composition for the antineoplastic taxane. Examples of such materials include biocompatible plasticizers, delivery agents, fillers and the like.

The present invention provides a number of methods of making the subject compositions. Examples of such methods include those described in the Exemplification below.

In certain embodiments, the subject compositions are in the form of microspheres. In other embodiments, the subject compositions are in the form of nanospheres. In one aspect, the subject compositions of the present invention may be lyophilized or subjected to another appropriate drying technique such as spray drying and subsequently rehydrated for ready use.

In another aspect, the present invention is directed to methods of using the subject polymer compositions for prophylactic or therapeutic treatment. In certain instances, the subject compositions may be used to prevent a disease or condition. In certain embodiments, use of certain of the subject compositions, which release in a sustained manner an antineoplastic taxane, allow for different treatment regimens than are possible with other modes of administration of such therapeutic agent.

In another aspect, the efficacy of treatment using the subject compositions may be compared to treatment regimens known in the art in which the antineoplastic taxane is not encapsulated within a subject polymer. For example, treatment with a subject composition is expected to result in fewer hypersensitivity reactions than treatment with an antineoplastic taxane, with or without premedication. Alternatively, as described below in the Exemplification, treatment with a subject composition results in an increase in the median survival rate in mice, and it is expected that the same will result in other mammals, and in particular humans.

In another aspect, the subject polymers may be used in the manufacture of a medicament for any number of uses, including for example treating any disease or other treatable condition of a patient. In still other aspects, the present invention is directed to a method for formulating polymers of the present invention in a pharmaceutically acceptable carrier.

In another aspect, the present invention may be spray dried and subsequently rehydrated for ready use or injected as powder using appropriate powder injecting device.

In other embodiments, this invention contemplates a kit including subject compositions, and optionally instructions for their use. Uses for such kits include, for example, therapeutic applications. In certain embodiments, the subject compositions contained in any kit have been lyophilized and require rehydration before use.

These embodiments of the present invention, other embodiments, and their features and characteristics, will be apparent from the description, drawings and claims that follow.

### BRIEF DESCRIPTION OF THE DRAWINGS

FIG. 1 shows the plasma concentrations of paclitaxel over time after subcutaneous administration of polymer microspheres containing differing doses of paclitaxel.

FIG. 2 depicts the increased lifespan of cancerous mice treated with microspheres containing paclitaxel relative to an untreated control group.

FIGS. 3 and 4 compare the efficacy of injections of paclitaxel solution with subcutaneous delivery of polymer microspheres incorporating paclitaxel at differing doses on the survival of cancerous mice.

FIG. 5 compares the efficacy of subcutaneous delivery of paclitaxel from polymer microspheres of various loadings.

FIG. 6 illustrates the plasma concentrations of paclitaxel over time after intravenous administration of paclitaxel as a solution or in a polymeric formulation.

FIG. 7 represents the plasma concentration of paclitaxel over time after subcutaneous injection of paclitaxel in polymeric microspheres.

FIG. 8 shows the efficacy of subcutaneous delivery of polymeric microspheres incorporating paclitaxel for a variety of administration schedules.

FIG. 9 presents results of an experiment comparing the efficacy of injections of paclitaxel solution with intravenous delivery of polymeric microspheres incorporating paclitaxel at differing doses on the survival of cancerous mice.

FIG. 10 depicts the plasma concentrations of paclitaxel that results from conventional administration or from administration of polymer microspheres incorporating paclitaxel at varying dosages.

### DETAILED DESCRIPTION OF THE INVENTION

1. Overview

The present invention relates to pharmaceutical compositions for the delivery of paclitaxel, docetaxel, or analogs thereof, e.g., for the treatment of cancer or other hyperproliferative disorders, i.e., conditions contributed to or caused by unwanted cell proliferation, or for treatment of inflammatory disorders, such as arthritis. In certain embodiments, biocompatible and optionally biodegradable polymers may be used to allow for sustained release of an encapsulated antineoplastic taxane. The present invention also relates to methods of administering such pharmaceutical compositions, e.g., as part of a treatment regimen, for example, subcutaneously, intravenously, or intramuscularly.

In certain aspects, the pharmaceutical compositions, upon contact with body fluids including blood, spinal fluid, lymph or the like, release the encapsulated drug over a sustained or extended period (as compared to the release from an isotonic saline solution). Such a system may result in prolonged delivery (over, for example, 2 to 4,000 hours, preferably 4 to 1500 hours) of effective amounts (e.g., 0.00001 mg/kg/hour to 10 mg/kg/hour) of the drug. This dosage form may be administered as is necessary depending on the subject being treated, the severity of the affliction, the judgment of the prescribing physician, and the like.

2. Definitions

For convenience, before further description of the present invention, certain terms employed in the specification, examples, and appended claims are collected here. These definitions should be read in light of the remainder of the disclosure and understood as by a person of skill in the art.

The term “antineoplastic” is art-recognized, and describes therapeutic agents including agents that prevent the development, maturation, or spread of cells characterized by abnormal malignant growth, e.g., for treating or preventing cancer. Preferably, an antineoplastic agent used in a composition of the invention as effective or more effective than paclitaxel or docetaxel, or is at least within an order of magnitude as effective as paclitaxel or docetaxel, e.g., has an ED₅₀ less than ten times the ED₅₀ of paclitaxel or docetaxel.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1M NaOH at 37° C. until complete degradation is observed. The solution is then neutralized with 1M HCl. About 200 μL of various concentrations of the degraded sample products are placed in 96-well tissue culture plates and seeded with human gastric carcinoma cells (GT3TKB) at 10⁴/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to side chain or that connects a side chain to the polymer backbone. For example, a therapeutic agent or other chemical moiety attached as a side chain to the polymer backbone may be released by biodegradation. In certain embodiments, one or the other or both generally types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments, if the biodegradable polymer also has a therapeutic agent or other material associated with it, the biodegradation rate of such polymer may be characterized by a release rate of such materials. In such circumstances, the biodegradation rate may depend on not only the chemical identity and physical characteristics of the polymer, but also on the identity of any such material incorporated therein.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, lymph or the like, the polymer matrices (formulated as provided herein and otherwise as known to one of skill in the art) may undergo gradual degradation (e.g., through hydrolysis) with concomitant release of any material incorporated therein, e.g., paclitaxel, for a sustained or extended period (as compared to the release from a bolus). This release may result in prolonged delivery of therapeutically effective amounts of any incorporated therapeutic agent. Sustained release will vary in certain embodiments as described in greater detail below.

The term “delivery agent” is an art-recognized term, and includes molecules that facilitate the intracellular delivery of a therapeutic agent or other material. Examples of delivery agents include: sterols (e.g., cholesterol) and lipids (e.g., a cationic lipid, virosome or liposome).

The term “microspheres” is art-recognized, and includes substantially spherical colloidal structures, e.g., formed from biocompatible polymers such as subject compositions, having a size ranging from about one or greater up to about 1000 microns. In general, “microcapsules”, also an art-recognized term, may be distinguished from microspheres, because microcapsules are generally covered by a substance of some type, such as a polymeric formulation. The term “microparticles” is art-recognized, and includes microspheres and microcapsules, as well as structures that may not be readily placed into either of the above two categories, all with dimensions on average of less than 1000 microns. If the structures are less than about one micron in diameter, then the corresponding art-recognized terms “nanosphere,” “nanocapsule,” and “nanoparticle” may be utilized. In certain embodiments, the nanospheres, nancapsules and nanoparticles have an average diameter of about 500, 200, 100, 50 or 10 nm.

A composition comprising microspheres may include particles of a range of particle sizes. In certain embodiments, the particle size distribution may be uniform, e.g., within less than about a 20% standard deviation of the median volume diameter, and in other embodiments, still more uniform or within about 10% of the median volume diameter.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is an art-recognized term which includes curing as well as ameliorating at least one symptom of any condition or disease.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as cancer, an infectious disease, or other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

The phrases “systemic administration,” “administered systemically,” “peripheral administration” and “administered peripherally” are art-recognized, and include the administration of a subject composition or other material other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of the therapeutic agent that, when incorporated into a polymer of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

In certain embodiments, a therapeutically effective amount of a taxane, such as paclitaxel, docetaxel, or an analog thereof, for in vivo use will likely depend on a number of factors, including: the rate of release of the agent from the polymer matrix, which will depend in part on the chemical and physical characteristics of the polymer; the identity of the agent; the mode and method of administration; and any other materials incorporated in the polymer matrix in addition to the taxane.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The terms “incorporated” and “encapsulated” are art-recognized when used in reference to a therapeutic agent, or other material and a polymeric composition, such as a composition of the present invention. In certain embodiments, these terms include incorporating, formulating or otherwise including such agent into a composition which allows for sustained release of such agent in the desired application. The terms may contemplate any manner by which a therapeutic agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved, in the polymer. Any form of encapsulation or incorporation is contemplated by the present invention, in so much as the sustained release of any encapsulated therapeutic agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyl trihexyl citrate (c. 20 weight percent or less), butyl benzyl phthalate, dibutyl phthalate, dioctylphthalate, n-butyryl tri-n-hexyl citrate, diethylene glycol dibenzoate (c. 20 weight percent or less) and the like.

“Small molecule” is an art-recognized term. In certain embodiments, this term refers to a molecule which has a molecular weight of less than about 2000 amu, or less than about 1000 amu, and even less than about 500 amu.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas:

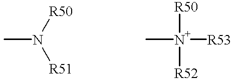

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

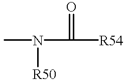

wherein R50 is as defined above, and R54 represents a hydrogen, an alkyl, an alkenyl or —(CH₂)ₘ—R6 1, where m and R61 are as defined above.

The term “amido” is art recognized as an amino-substituted carbonyl and includes a moiety that may be represented by the general formula:

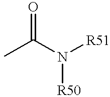

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

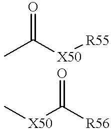

wherein X50 is a bond or represents an oxygen or a sulfur, and R55 represents a hydrogen, an alkyl, an alkenyl, —(CH₂)ₘ—R61or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and includes a moiety that may be represented by the general formula:

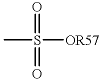

in which R57 is an electron pair, hydrogen, alkyl, cycloalkyl, or aryl.

The term “sulfate” is art recognized and includes a moiety that may be represented by the general formula:

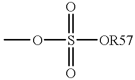

in which R57 is as defined above.

The term “sulfonamido” is art recognized and includes a moiety that may be represented by the general formula:

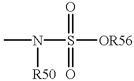

in which R50 and R56 are as defined above.

The term “sulfamoyl” is art-recognized and includes a moiety that may be represented by the general formula:

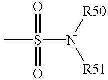

in which R50 and R51 are as defined above.

The term “sulfonyl” is art recognized and includes a moiety that may be represented by the general formula:

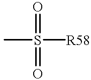

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

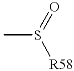

in which R58 is defined above.

The term “phosphoramidite” is art recognized and includes moieties represented by the general formulas:

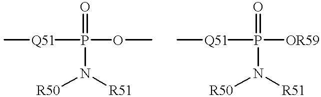

wherein Q51, R50, R51 and R59 are as defined above.

The term “phosphonamidite” is art recognized and includes moieties represented by the general formulas:

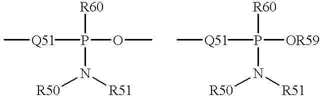

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art recognized and includes an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl,p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described above include such materials which otherwise correspond thereto, and which have the same general properties thereof (e.g., biocompatible, antineoplastic), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

3. Exemplary Subject Compositions

A. Taxanes and Other Therapeutic Molecules

A subject composition may comprise paclitaxel, docetaxel or an analog thereof. Paclitaxel and docetaxel share a common framework, and differ primarily in the substituents at two sites on this framework, shown as R1 and R2 in Formula I below:

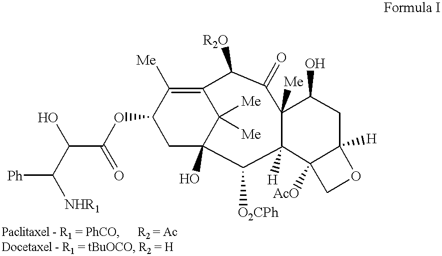

Thus, in one embodiment, a therapeutic composition of the invention comprises a compound of the above formula, wherein R1 is an acyl group or R1—N taken together comprise a carbamyl group (O—C(═O)—N), and R2 is H or an acyl group. In preferred embodiments, R1 comprises between 2 and 12 carbon atoms, preferably between 4 and 9 carbon atoms. In preferred embodiments, R2 is H or an acyl group having between 2 and 8 carbons, preferably between 2 and 4 carbons. In certain embodiments, the therapeutic agent is docetaxel or paclitaxel.

In another embodiment, a therapeutic composition of the present invention includes an antineoplastic compound having a structure of Formula II:

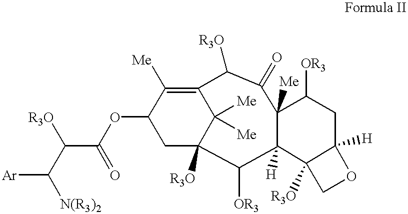

wherein, independently for each occurrence:

Ar represents a substituted or unsubstituted aryl or heteroaryl group; and

R3, each independently, represents H, alkyl, acyl, alkoxycarbonyl, aryloxycarbonyl, aminocarbonyl or sulfonyl.

In certain embodiments, at least one R3 is bound to nitrogen is H or alkyl. In certain embodiments, at least one R3 bound to nitrogen is acyl, alkoxycarbonyl, aryloxycarbonyl, aminocarbonyl, or sulfonyl. In certain embodiments, when R3 is bound to oxygen, R3 is selected from H, alkyl, acyl, aminocarbonyl, alkoxycarbonyl, or aryloxycarbonyl. Preferably, an R3 is selected to be sterically similar to a corresponding substituent on paclitaxel or docetaxel, i.e., contains a number of carbon atoms within four of the number of carbon atoms in a similarly situated substituent of paclitaxel or docetaxel. For example, the benzoate ester of paclitaxel may be exchanged for a tosyl (p-toluenesulfonyl) ester, a cyclohexyl carbamate, or a tetrachlorobenzocyclopentanol carbonate, or a hydroxyl of docetaxel may be exchanged for an ethyl ether, a methylsulfonate ester, or a 2-hydroxyethyl carbamate.

In yet another embodiment, a therapeutic composition of the present invention includes an antineoplastic compound having a structure of Formula III:

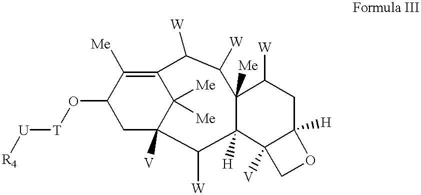

wherein, independently for each occurrence:

V, each independently, represents H, hydroxy, lower alkoxy, or a small ester (e.g., less than 4 carbons);

W, each independently, represents H, hydroxy, carbonyl, amino, alkoxy, sulfhydryl, alkylthio, ester, acylamino, carbamate, sulfonate, carbonate, or sulfoxide;

T represents —C(═O)—, —C(═S)—, —SO₂—, or —SO—;

U is absent or represents NH, S, or O; and

R4 represents a substituted aralkyl.

In certain embodiments, at least one occurrence of W or R4 includes a moiety, such as an oligopeptide or an oligosaccharide, that improves the bioavailability and/or solubility of the taxane. In certain embodiments, the therapeutic compound is formulated as a prodrug, e.g., at least one occurrence of W or R4 includes a moiety capable of being hydrolyzed and cleaved from the molecule under physiological conditions. The hydrolyzable moiety may improve the bioavailability and/or solubility of the taxane. The prodrug form of the therapeutic compound may itself be inactive, provided that after cleavage of the hydrolyzable moiety, the resulting compound is antineoplastic. In certain embodiments, at least one occurrence of W or R4 includes a bond to a polymer, preferably a biocompatible and/or biodegradable polymer. The bond to the polymer may be hydrolyzable under physiologic conditions.

In certain embodiments, a therapeutic composition of the present invention includes an “antineoplastic taxane”, i.e., a compound which has a framework of Formula IV:

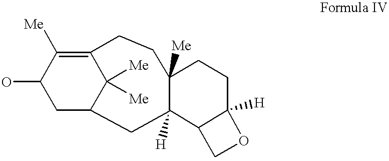

wherein, such framework bears sufficient substituents disposed at unspecified positions, as valence allows, such that the resulting compound has antineoplastic activity. In certain embodiments, such a compound is formed by chemically modifying paclitaxel or 10-deacetylbaccatin III, a naturally occurring compound which has the structure:

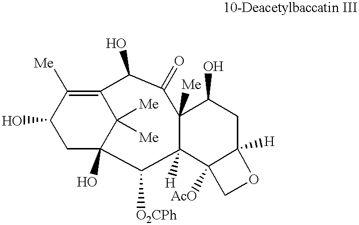

A variety of such antineoplastic derivatives are known in the art, and may be employed in the subject compositions and methods without departing from the spirit or scope of the present invention.

B. Polymers

A variety of polymers may be used in the subject invention. Both non-biodegradable and biodegradable polymers may be used in the subject invention, although biodegradable polymers are preferred. As discussed below, the choice of polymer will depend in part on a variety of physical and chemical characteristics of such polymer and the use to which such polymer may be put.

Representative natural polymers include proteins, such as zein, modified zein, casein, gelatin, gluten, serum albumin, or collagen, and polysaccharides, such as cellulose, dextrans, hyaluronic acid, and polymers of alginic acid.

Representative synthetic polymers include polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyphosphates and polyurethanes.

Synthetically modified natural polymers include alkyl celluloses, hydroxyalkyl celluloses, cellulose ethers, cellulose esters, and nitrocelluloses. Other like polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

Representative biodegradable polymers include polylactide, polyglycolide, polycaprolactone, polycarbonate, poly(phosphoesters), polyanhydride, polyorthoesters, and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins.

All of the subject polymers may be provided as copolymers or terpolymers. These polymers may be obtained from chemical suppliers or else synthesized from monomers obtained from these suppliers using standard techniques.

In addition to the listing of polymers above, polymers having phosphorus linkages may be used in the subject invention. Exemplary phosphorus linkages in such polymers include, without limitation, phosphonamidite, phosphoramidite, phosphorodiamidate, phosphomonoester, phosphodiester, phosphotriester, phosphonate, phosphonate ester, phosphorothioate, thiophosphate ester, phosphinate or phosphite. Certain of such polymers may be biodegradable, biocompatible or both.

The structure of certain of the foregoing polymers having phosphorus linkages may be identified as follows. The term “polymer having phosphorous-based linkages” is used herein to refer to polymers in which the following substructure is present at least a multiplicity of times in the backbone of such polymer:

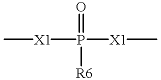

wherein, independently for each occurrence of such substructure:

X1, each independently, represents —O— or —N(R5)—;

R5 represents —H, aryl, alkenyl or alkyl; and

R6 is any non-interfering substituent,

wherein such substructure is responsible in part for biodegradability properties, if any, observed for such polymer in vitro or in vivo. In certain embodiments, R6 may represent an alkyl, aralkyl, alkoxy, alkylthio, or alkylamino group.

In certain embodiments, such a biodegradable polymer is non-naturally occurring, i.e., a man-made product with no natural source. In other embodiments, R6 is other than —OH or halogen, e.g., is alkyl, aralkyl, aryl, alkoxy, aralkyoxy or aryloxy. In still other embodiments, the two X1 moieties in such substructure are the same. For general guidance, when reference is made to the “polymer backbone chain” or the like of a polymer, with reference to the above structure, such polymer backbone chain comprises the motif [—X1—P—X1—]. In other polymers, the polymer backbone chain may vary as recognized by one of skill in the art.

By way of example, but not limitation, a number of representative polymers having phosphorus linkages are described in greater detail below. In certain embodiments, a polymer includes one or more monomeric units of Formula V:

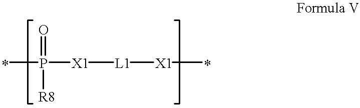

wherein, independently for each occurrence of such unit:

X1, each independently, represents —O— or —N(R7)—;

R7 represents —H, aryl, alkenyl or alkyl;

L1 is described below;

R8 represents, for example, —H, alkyl, —O-alkyl, —O-cycloalkyl, aryl, —O-aryl, heterocycle, —O-heterocycle, —N(R9)R10 and other examples presented below;

R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;

m represents an integer in the range of 0-10, preferably 0-6; and

R11 represents —H, alkyl, aryl, cycloalkyl, cycloalkenyl, heterocycle or polycycle.

L1 may be any chemical moiety as long as it does not materially interfere with the polymerization, biocompatibility or biodegradation (or any combination of those three properties) of the polymer, wherein a “material interference” or “non-interfering substituent” is understood to mean: (i) for synthesis of the polymer by polymerization, an inability to prepare the subject polymer by methods known in the art or taught herein, (ii) for biocompatibility, a reduction in the biocompatibility of the subject polymer so as to make such polymer impracticable for in vivo use; and (iii) for biodegradation, a reduction in the biodegradation of the subject polymer so as to make such polymer impracticable for biodegradation.

In certain embodiments, L1 is an organic moiety, such as a divalent branched or straight chain or cyclic aliphatic group or divalent aryl group, with in certain embodiments, from 1 to about 20 carbon atoms. In certain embodiments, L1 represents a moiety between about 2 and 20 atoms selected from carbon, oxygen, sulfur, and nitrogen, wherein at least 60% of the atoms are carbon. In certain embodiments, L1 may be an alkylene group, such as methylene, ethylene, 1,2-dimethylethylene, n-propylene, isopropylene, 2,2-dimethylpropylene, n-pentylene, n-hexylene, n-heptylene; an alkenylene group such as ethenylene, propenylene, 2-(3-propenyl)-dodecylene; and an alkynylene group such as ethynylene, proynylene, 1-(4-butynyl)-3-methyldecylene; and the like. Such unsaturated aliphatic groups may be used to cross-link certain embodiments of the present invention.

Further, L1 may be a cycloaliphatic group, such as cyclopentylene, 2-methylcyclopentylene, cyclohexylene, cyclohexylenedimethylene, cyclohexenylene and the like. L1 may also be a divalent aryl group, such as phenylene, benzylene, naphthalene, phenanthrenylene and the like. Further, L1 may be a divalent heterocyclic group, such as pyrrolylene, furanylene, thiophenylene, alkylyene-pyrrolylene-alkylene, pyridinylene, pyrimidinylene and the like.

Other examples of L1 may include any of the polymers listed above, including the biodegradable polymers listed above, and in particular polylactide, polyglycolide, polycaprolactone, polycarbonate, polyethylene terephthalate, polyanhydride and polyorthoester, and polymers of ethylene glycol, propylene glycol and the like. Embodiments containing such polymers for L1 may impart a variety of desired physical and chemical properties.

The foregoing, as with other moieties described herein, may be substituted with a non-interfering substituent, for example, a hydroxy-, halogen-, or nitrogen-substituted moiety.

R8 represents hydrogen, alkyl, cycloakyl, —O-alkyl, —O-cycloalkyl, aryl, —O-aryl, heterocycle, —O-heterocycle, or —N(R9)R10. Examples of possible alkyl R8 groups include methyl, ethyl, n-propyl, i-propyl, n-butyl, tert-butyl, —C₈H₁₇ and the like groups; and alkyl substituted with a non-interfering substituent, such as hydroxy, halogen, alkoxy or nitro; corresponding alkoxy groups.

When R8 is aryl or the corresponding aryloxy group, it typically contains from about 5 to about 14 carbon atoms, or about 5 to about 12 carbon atoms, and optionally, may contain one or more rings that are fused to each other. Examples of particularly suitable aromatic groups include phenyl, phenoxy, naphthyl, anthracenyl, phenanthrenyl and the like.

When R8 is heterocyclic or heterocycloxy, it typically contains from about 5 to about 14 ring atoms, alternatively from about 5 to about 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,2-pyran, 1,4-pyran, 1,2-pyrone, 1,4-pyrone, 1,2-dioxin, 1,3-dioxin, pyridine, N-alkyl pyridinium, pyridazine, pyrimidine, pyrazine, 1,3,5-triazine, 1,2,4-triazine, 1,2,3-triazine, 1,2-oxazine, 1,3-oxazine, 1,4-oxazine, o-isoxazine, p-isoxazine, 1,2,5-oxathiazine, 1,2,6-oxathiazine, 1,4,2-oxadiazine, 1,3,5-oxadiazine, azepine, oxepin, thiepin, indene, isoindene, benzofuran, isobenzofuran, thionaphthene, isothionaphthene, indole, indolenine, 2-isobenzazole, isoindazole, indoxazine, benzoxazole, anthranil, 1,2-benzopyran, 1,2-benzopyrone, 1,4-benzopyrone, 2,1-benzopyrone, 2,3-benzopyrone, quinoline, isoquinoline, 12,-benzodiazine, 1,3-benzodiazine, naphthyridine, pyrido-[3,4-b]-pyridine, pyrido-[3,2-b]-pyridine, pyrido-[4,3-b]-pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In certain embodiments, when R8 is heterocyclic or heterocycloxy, it is selected from the group consisting of furan, pyridine, N-alkylpyridine, 1,2,3- and 1,2,4-triazoles, indene, anthracene and purine rings.

In certain embodiments, R8 is an alkyl group, an alkoxy group, a phenyl group, a phenoxy group, a heterocycloxy group, or an ethoxy group.

In still other embodiments, R8, such as an alkyl, may be conjugated to a bioactive substance to form a pendant drug delivery system.

In certain embodiments, the number of monomeric units in Formula V and other subject formulas that make up the subject polymers ranges over a wide range, e.g., from about 5 to 25,000 or more, but generally from about 100 to 5000, or 10,000.

Alternatively, in other embodiments, n may be about 10, 25, 50, 75, 100, 150, 200, 300 or 400.

In Formula V and other formulas herein, “*” represents other monomeric units of the subject polymer, which may be the same or different from the unit depicted in the formula in question, or a chain terminating group, by which the polymer terminates. Examples of such chain terminating groups include monofunctional alcohols and amines.

In another aspect, the polymeric compositions of the present invention include one or more recurring monomeric units represented in general Formula VI:

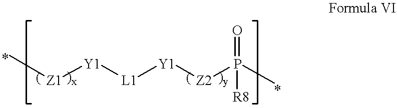

wherein Z1 and Z2, respectively, for each independent occurrence is:

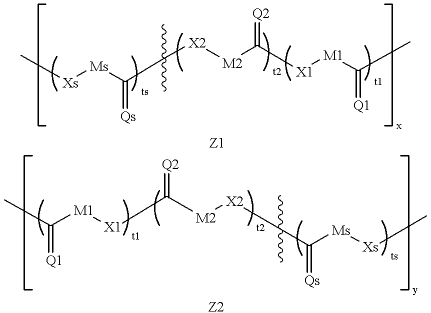

wherein, independently for each occurrence set forth above:

Q1, Q2. . . Qs, each independently, represent O or N(R1);

X1, X2. . . Xs, each independently, represent —O— or —N(R1);

the sum of t1, t2. . . ts is an integer and at least one or more;

Y1 represents —O—, —S— or —N(R7)—;

x and y are each independently integers from 1 to about 1000 or more;

L1 and M1, M2. . . Ms each independently, represent the moieties discussed below; and

the other moieties are as defined above.

M1, M2. . . Ms (collectively, M) in Formula VI are each independently any chemical moiety that does not materially interfere with the polymerization, biocompatibility or biodegradation (or any combination of those three properties) of the subject polymer. For certain embodiments, M in the formula are each independently: (i) a branched or straight chain aliphatic or aryl group having from 1 to about 50 carbon atoms, or (ii) a branched or straight chain, oxa-, thia-, or aza-aliphatic group having from 1 to about 50 carbon atoms, both optionally substituted. In certain embodiments, the number of such carbon atoms does not exceed 20. In other embodiments, M may be any divalent aliphatic moiety having from 1 to about 20 carbon atoms, including therein from 1 to about 7 carbon atoms.

M may include an aromatic or heteroaromatic moiety, optionally with non-interfering substituents. In certain embodiments, none of the atoms (usually but not always C) that form the cyclic ring that gives rise to the aromatic moiety are part of the polymer backbone chain.

Specifically, when M is a branched or straight chain aliphatic group having from 1 to about 20 carbon atoms, it may be, for example, an alkylene group such as methylene, ethylene, 1-methylethylene, 1,2-dimethylethylene, n-propylene, trimethylene, isopropylene, 2,2-dimethylpropylene, n-pentylene, n-hexylene, n-heptylene, n-octylene, n-nonylene, n-decylene, n-undecylene, n-dodecylene, and the like; an alkenylene group such as n-propenylene, 2-vinylpropylene, n-butenylene, 3-thexylbutylene, n-pentenylene, 4-(3-propenyl)hexylene, n-octenylene, 1-(4-butenyl)-3-methyldecylene, 2-(3-propenyl)dodecylene, hexadecenylene and the like; an alkynylene group, such as ethynylene, propynylene, 3-(2-ethynyl)pentylene, n-hexynylene, 2-(2-propynyl)decylene, and the like; or any alkylene, alkenylene or alkynylene group, including those listed above, substituted with a materially non-interfering substituent, for example, a hydroxy, halogen or nitrogen group, such as 2-chloro-n-decylene, 1-hydroxy-3-ethenylbutylene, 2-propyl-6-nitro-10-dodecynylene, and the like. Other M of the present invention include —(CH₂)₃—, —(CH₂)₅— and —(CH₂)₂OCH₂—.

When M is a branched or straight chain oxaaliphatic group having from 1 to about 20 carbon atoms, it may also be, for example, a divalent alkoxylene group, such as ethoxylene, 2-methylethoxylene, propoxylene, butoxylene, pentoxylene, dodecyloxylene, hexadecyloxylene, and the like. When M is a branched or straight chain oxaaliphatic group, it may have the formula —(CH₂)ₐ—O—(CH₂)_{b}— wherein each of a and b, independently, is about 1 to about 7.

When M is a branched or straight chain oxaaliphatic group having from 1 to about 20 carbon atoms, it may also be, for example, a dioxxaalkylene group such as dioxymethylene, dioxyethylene, 1,3-dioxypropylene, 2-methoxy-1,3-dioxypropylene, 1,3-dioxy-2-methylpropylene, dioxy-n-pentylene, dioxy-n-octadecylene, methoxylene-methoxylene, ethoxylene-methoxylene, ethoxylene-ethoxylene, ethoxylene-1-propoxylene, butoxylene-n-propoxylene, pentadecyloxylene-methoxylene, and the like. When M is a branched or straight chain, dioxyaliphatic group, it may have the formula —(CH₂)ₐ—O—(CH₂)_{b}—O—(CH₂)_{c}—, wherein each of a, b, and c is independently from 1 to about 7.

When M is a branched or straight chain thiaaliphatic group, the group may be any of the preceding oxaaliphatic groups wherein the oxygen atoms are replaced by sulfur atoms.

When M is a branched or straight chain, aza-aliphatic group having from 1 to about 20 carbon atoms, it may be a divalent group such as —CH₂NH—, —(CH₂)₂N—, —CH₂(C₂H₅)N—, —n—C₄H₉NH—, —t—C₄H₉NH—, —CH₂(C₃H₇)N—, —C₂H₅(C₂H₅)N—, —CH₂(C₈H₁₇)N—, —CH₂NHCH₂—, —(CH₂)₂NCH₂—, —CH₂(C₂H₅)NCH₂CH₂—, —n—C₄H₉NHCH₂—, —t—C₄H₉NHCH₂CH₂—, —CH₂(C₃H₇)N(CH₂)₄—, —C₂H₅(C₂H₅)NCH₂—, —CH₂(C₈H₁₇)NCH₂CH₂—, and the like. When M is a branched or straight chain, amino-aliphatic group, it may have the formula —(CH₂)ₐNR1— or —(CH₂)ₐN(R1)(CH₂)_{b}— where R1 is —H, aryl, alkenyl or alkyl and each of a and b is independently from about 1 to about 7.

x and y of Formula VI each independently represent integers in the range of about 1 to about 1000, e.g., about 1, about 10, about 20, about 50, about 100, about 250, about 500, about 750, about 1000, etc.

For Formula VI, the average molar ratio of (x or y):L1, assuming ts is equal to one, may vary greatly, typically between about 75:1 and about 2:1. In certain embodiments, the average molar ratio of (x or y):L1, when ts is equal to one, is about 10:1 to about 4:1, and preferably about 5:1. The molar ratio of x:y may also vary; typically, such ratio is about 1. Other possible embodiments may have ratios of 0.1, 0.25, 0.5, 0.75, 1.5, 2, 3, 4, 10 and the like.

A number of different polymer structures are contemplated by Formula VI. For example, in certain polymers exemplified by Formula VI, when the sum of t1, t2. . . ts equals one for each of Z1 and Z2 and Q, M and X for each subunit ts are the same, then Formula VI becomes the following Formula VIa:

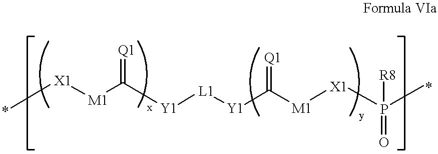

In certain embodiments of Formula VIa (and other subject formulas), x and y may be even integers.

The above Formula VI (and all of the subject formulae and polymers) encompass a variety of different polymer structures, including block copolymers, random copolymers, random terpolymers and segmented block copolymers and terpolymers. Additional structures for Z of subject monomeric units are set forth below, which exemplify in part the variety of structures contemplated by the present invention:

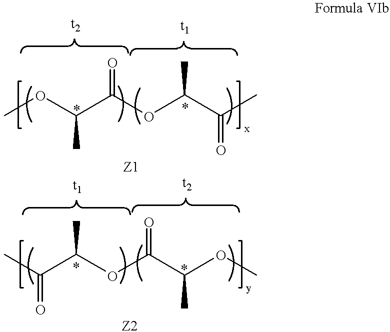

In Formula VIb (and other formulas described below), there may be more ts subunits depicted of the same molecular identity of those depicted in the formulas. For example, in Formula VIb, subunits t₁ and t₂ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₂), such that both subunits may be present in approximately the same amount, or in differing amounts, which may differ slightly or be highly disparate, e.g., one subunit is present nearly to the exclusion of the other. In certain embodiments, the chiral centers of each subunit may be the same or different and may be arranged in an orderly fashion or in a random sequence in each of Z1 and Z2.

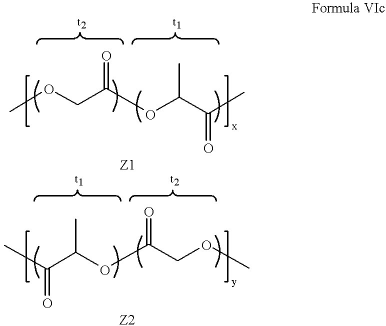

In certain embodiments of Formula VIc, the sum of the number of ts subunits in each of Z1 and Z2 is an even integer. As in other examples of Z1 and Z2, such as described above for Formula VIb, the ts subunits may be distributed randomly or in an ordered arrangement in each of Z1 or Z2.

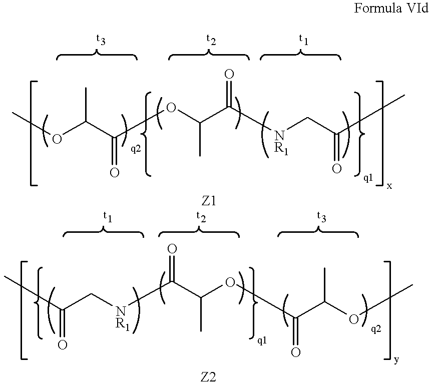

In Formula VId, the subunit q1 is comprised of two ts subunits, which may be repeated and arranged as described above for Formula VIb. In certain embodiments, q2 is an even integer, and in other embodiments, the subunits q1 and q2 may be distributed randomly or in an ordered pattern in each of Z1 and Z2. For example, subunits q₁ and q₂ may be repeated in a sequence, e.g., alternating, in blocks (which may themselves repeat), or in any other pattern or random arrangement. Each subunit may repeat any number of times, and one subunit (e.g., q₁) may occur with substantially the same frequency, more often, or less often than another subunit (e.g., q₂), such that both subunits may be present in approximately the same amount, or in differing amounts, which may differ slightly or be highly disparate, e.g., one subunit is present nearly to the exclusion of the other.

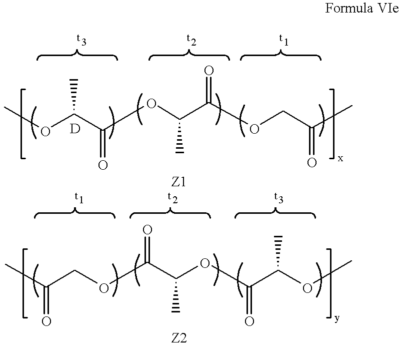

In certain embodiments of Formula VIe, the sum of the ts subunits for each of Z1 and Z2 is an even integer. In other embodiments, the each of the subunits t₁, t₂, and t₃ may be distributed randomly or in an ordered arrangement in each of Z1 and Z2. For example, in Formula VIe, subunits t₁, t₂, and t₃ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₃), such that the three subunits may be present in approximately the same amount, or in differing amounts, which may differ slightly or be highly disparate, e.g., two subunits are present nearly to the exclusion of the third.

In certain embodiments of Formula VI, in which Q, M and X for each subunit are the same, Q1 represents O, M represents a lower alkylene group, and X1 represents O or S, preferably O. For example, M may represent —CH(CH₃)— to result in a polymer of Formula VI having a structure represented in Formula VIf:

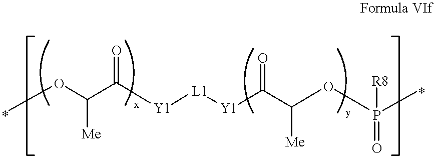

In certain embodiments of Formula VIf, as further described in the Exemplification below, L1 represents a lower alkylene chain, such as ethylene, propylene, etc. In certain embodiments, all Y1's represent O. In certain embodiments, R8 represents —O-lower alkyl, such as —OEt.

In certain embodiments of polymers depicted by Formula VI, the chirality of each subunit is identical, whereas in other embodiments, the chirality is different. By way of example but not limitation, in Formula VIb above, if the chiral centers of all of the subunits are D-enantiomers or L-enantiomers, then the monomeric unit is effectively equivalent to D-lactic acid or L-lactic acid, respectively, thereby giving rise to a region similar to poly(D-lactic acid) or poly (L-lactic acid), respectively. Conversely, if the two subunits in Formula VIb are comprised of alternating D- and L-enantiomers (e.g., one unit of D-enantiomer, one unit of L-enantiomer, etc.), then the resulting polymeric region is analogous to poly(meso-lactic acid) (i.e., a polymer formed by polymerization of meso-lactide).

Finally, in certain embodiments of the monomeric units set forth in Formula VI, in which the entire polymer may or may not be composed of such units, the following moieties for Y1, L1, R8 Qs, Xs and Ms may be used (with a variety of different x and y being possible):

| Abbreviation | All Y1's | L1 | R8 |
|---|---|---|---|
| L-PL(EG)EOP | O | —CH₂CH₂— | —OCH₂CH₃ |
| L-PL(EG)HOP | O | —CH₂CH₂— | —O(CH₂)₅CH₃ |
| D,L-PL(EG)EOP* | O | —CH₂CH₂— | —OCH₂CH₃ |
| D,L-PL(PG)EOP* | O | —CH₂(CH₃)CH₂— | —OCH₂CH₃ |
| D-PL(PG)EOP | O | —CH₂(CH₃)CH₂— | —OCH₂CH₃ |
| L-PL(PG)EOP | O | —CH₂(CH₃)CH₂— | —OCH₂CH₃ |
| D,L-PL(HD)EOP* | O | 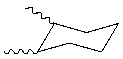 | —OCH₂CH₃ |
| D,L-PL(PG)HOP* | O | —CH₂(CH₃)CH₂— | —O(CH₂)₅CH₃ |
| D,L-PL(PG)EP* | O | —CH₂(CH₃)CH₂— | —CH₂CH₃ |

| Abbreviation | All Qs | All Xs | M1 | M2 |
|---|---|---|---|---|
| L-PL(EG)EOP | O | O | —CH(CH₃)— (L) | N/A |
| L-PL(EG)HOP | O | O | —CH(CH₃)— (L) | N/A |
| D,L-PL(EG)EOP* | O | O | —CH(CH₃)— (L or D) | —CH(CH₃)— (D or L) |
| D,L-PL(PG)EOP* | O | O | —CH(CH₃)— (L or D) | —CH(CH₃)— (D or L) |
| D-PL(PG)EOP | O | O | —CH(CH₃)— (D) | N/A |
| L-PL(PG)EOP | O | O | —CH(CH₃)— (L) | N/A |
| D,L-PL(HD)EOP* | O | O | —CH(CH₃)— (L or D) | —CH(CH₃)— (L or D) |
| D,L-PL(PG)HOP* | O | O | —CH(CH₃)— (L or D) | —CH(CH₃)— (L or D) |
| D,L-PL(PG)EP* | O | O | —CH(CH₃)— (L or D) | —CH(CH₃)— (L or D) |

*For D,L-PL(EG)EOP*, D,L-PL(PG)EOP*, D,L-PL(HD)EOP*, D,L-PL(PG)HOP*, and D,L-PL(PG)EP*, if the chiral carbon of M1 has configuration L, then M2 will have

configuration D, and vice-versa. The order of the chiral centers in each subunit M1 and M2 for each Z1 and Z2 will be in random order.

In addition to the particular chiral version of the subject polymers described in the above table, polymers in which the chirality of MS varies in each subunit M in the subject polymers are also possible. For instance, referring to D,L-PL(EG)EOP by example, a random order of D and L, in varying amounts, are possible for this polymer. In contrast, the table sets forth one such example in which a D and L chiral M are always adjacent, in equal amounts, but that need not always be the case.

In another embodiment of the present invention, the polymeric compositions of the present invention include one or more recurring monomeric units represented in general Formula VII:

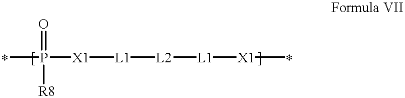

wherein, independently for each occurrence:

L2 is a divalent organic group as described in greater detail below; and

the other moieties are as defined as above.

In Formula VII, L2 may be a divalent, branched or straight chain aliphatic group, a cycloaliphatic group, or a group of the formula:

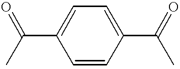

Specific examples of particular divalent, branched or straight chain aliphatic groups include an alkylene group with 1 to 7 carbon atoms, such as 2-methylpropylene or ethylene. Specific examples of cycloaliphatic groups include cycloalkylene groups, such as cyclopentylene, 2-methylcyclopentylene, cyclohexylene and 2-chloro-cyclohexylene; cycloalkenylene groups, such as cyclohexenylene; and cycloalkylene groups having fused or bridged additional ring structures, such as tetralinylene, decalinylene and norpinanylene; or the like.

In certain embodiments of the monomeric units set forth in Formula VII, in which the entire polymer may or may not be composed of such units, the following moieties for X1, L1 and R8 may be used:

| Abbreviation | All X1 | All L1 | L2 | R8 |
|---|---|---|---|---|
| P(trans-CHDM/HOP) | O | —CH₂— | 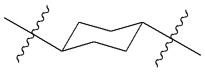 | —O(CH₂)₅CH₃ |
| P(cis- and trans- | O | —CH₂— | mixture of trans-1,4- | —O(CH₂)₅CH₃ |
| CHDM/HOP) | | | cyclohexyl and | |
| | | | 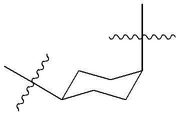 | |
| P(trans-CHDM/BOP) | O | —CH₂— | trans-1,4-cyclohexyl | —O(CH₂)₃CH₃ |
| P(trans-CHDM/EOP) | O | —CH₂— | trans-1,4-cyclohexyl | —OCH₂CH₃ |

In another embodiment of the present invention, the polymeric compositions of the present invention include one or more recurring monomeric units represented in general Formula VIII:

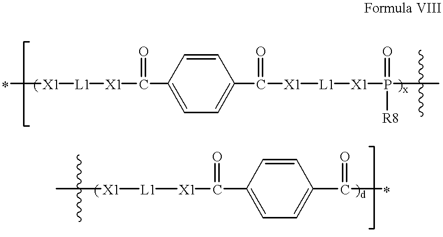

wherein, independently for each occurrence, d is equal to one or more, and optionally two, x is equal to or greater than one, and all of the other moieties are as defined above. In certain embodiments of Formula VIII, each of L1 independently may be an alkylene group, a cycloaliphatic group, a phenylene group or a divalent group of the formula:

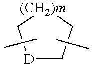

wherein D is O, N or S and m is 0 to 3. Alternatively, L1 is a branched or straight chain alkylyene group having from 1 to 7 carbon atoms, such as a methylene, ethylene, n-propylene, 2-methylpropylene, 2,2′-dimethylpropylene group and the like.

In certain embodiments of the monomeric units set forth in Formula VIII, in which the entire polymer may or may not be composed of such units, the following moieties for X1, L1 and R8 may be used (with a variety of different x possible for each example with d preferably equal to two):

| Abbreviation | All X1 | All L1 | R8 |
|---|---|---|---|
| P(BHET-EOP/TC) | O | —CH₂CH₂— | —OCH₂CH₃ |
| P(BHDPT-EOP/TC) | O | —CH₂CH(CH₃)₂CH₂— | —OCH₂CH₃ |
| P(BHDPT-HOP/TC) | O | —CH₂CH(CH₃)₂CH₂— | —OC₆H₁₃ |
| P(BHPT-EOP/TC) | O | —CH₂CH₂CH₂— | —OCH₂CH₃ |
| P(BHMPT-EOP/TC) | O | CH₂CH₂(CH₃)CH₂— | —OCH₂CH₃ |

In Formula VIII, the aryl groups represented therein may be substituted with a non-interfering substituent, for example, a hydroxy-, halogen-, or nitrogen-substituted moiety.

Other phosphorus containing polymers which may be adapted for use in the subject invention are described in the art, including those described in U.S. Pat. Nos. 5,256,765 and 5,194,581; PCT publications WO 98/44020, WO 98/44021, and WO 98/48859; and U.S. applications Ser. Nos. 09/053,649, 09/053,648 and 09/070,204. For all of the above-identified groups, non-interfering substituents may also be present.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain. For example, the polymer having units of Formula V may consist of effectively only one type of such subunit, or alternatively two or more types of such subunits. In addition, the polymer may contain monomeric units other than those subunits represented by Formula V.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element, such as a subunit depicted in Formula V. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. For example, in some instances, a polymer may be composed of two different subunits that may be both represented by the generic Formula V, but which differ in their chemical identity. In certain embodiments, the polymers may have just a few percent, or even less (for example, about 5, 2.5, 1, 0.5, 0.1%) of the subunits having phosphorous-based linkages. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have molecular weights ranging from about 2000 or less to about 1,000,000 or more daltons, or alternatively about 10,000, 20,000, 30,000, 40,000, or 50,000 daltons, more particularly at least about 100,000 daltons, and even more specifically at least about 250,000 daltons or even at least 500,000 daltons. Number-average molecular weight (Mn) may also vary widely, but generally fall in the range of about 1,000 to about 200,000 daltons, preferably from about 1,000 to about 100,000 daltons and, even more preferably, from about 1,000 to about 50,000 daltons. Most preferably, Mn varies between about 8,000 and 45,000 daltons. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In certain embodiments, the intrinsic viscosities of the polymers generally vary from about 0.01 to about 2.0 dL/g in chloroform at 40° C., alternatively from about 0.01 to about 1.0 dL/g and, occasionally, from about 0.01 to about 0.5 dL/g.

The glass transition temperature (Tg) of the subject polymers may vary widely, and depend on a variety of factors, such as the degree of branching in the polymer components, the relative proportion of phosphorous-containing monomer used to make the polymer, and the like. When the article of the invention is a rigid solid, the Tg is often within the range of from about −10° C. to about 80° C., particularly between about 0 and 50° C. and, even more particularly between about 25° C. to about 35° C. In other embodiments, the Tg is preferably low enough to keep the composition of the invention flowable at body temperature. Then, the glass transition temperature of the polymer used in the invention is usually about 0 to about 37° C., or alternatively from about 0 to about 25° C.

In certains embodiments, substituents of the phosphorus atom, such as R8 in the above formulas, and other components of the subject polymers may permit additional inter-chain cross-linking by covalent or electrostatic interactions (including, for example, hydrogen-binding or the formation of salt bridges) by having a side chain of either of them appropriately substituted as discussed in greater detail below.

In other embodiments, the polymer composition of the invention may be a flexible or flowable material. By “flowable” is meant the ability to assume, over time, the shape of the space containing it at body temperature. This includes, for example, liquid compositions that are capable of being sprayed into a site; injected with a manually operated syringe fitted with, for example, a 23-gauge needle; or delivered through a catheter.

Also included by the term “flowable”, are highly viscous, “gel-like” materials at room temperature that may be delivered to the desired site by pouring, squeezing from a tube, or being injected with any one of the commercially available power injection devices that provide injection pressures greater than would be exerted by manual means alone for highly viscous, but still flowable, materials. When the polymer used is itself flowable, the polymer composition of the invention, even when viscous, need not include a biocompatible solvent to be flowable, although trace or residual amounts of biocompatible solvents may still be present.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methyl pyrrolidone, dimethylformamide, and dimethylsulfoxide.

C. Therapeutic Compositions

In part, a biodegradable therapeutic polymer composition of the present invention includes both: (a) paclitaxel, docetaxel, or an analog thereof, such as a compound of Formula I, II, III, or IV, and (b) a biocompatible and optionally biodegradable polymer, such as one having the recurring monomeric units shown in one of the foregoing formulas, or any other biocompatible polymer mentioned above or known in the art.

In addition to antineoplastic taxane, the subject compositions may contain a “drug”, “therapeutic agent”, “medicament” or “bioactive substance”, which are biologically, physiologically, or pharmacologically active substances that act locally or systemically in the human or animal body. Various forms of the medicaments or biologically active materials may be used which are capable of being released from the polymer matrix into adjacent tissues or fluids. They may be acidic, basic, or salts. They may be neutral molecules, polar molecules, or molecular complexes capable of hydrogen bonding. They may be in the form of ethers, esters, amides and the like, which are biologically activated when injected into the human or animal body. An antineoplastic taxane is also an example of a “bioactive substance.”

Any additional bioactive substance in a subject composition may vary widely with the purpose for the composition. The term bioactive agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Plasticizers and stabilizing agents known in the art may be incorporated in polymers of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results. For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluoses and polysaccharides, including mannitose and sucrose, may be used in certain embodiments in the present invention.

In other embodiments, spheronization enhancers facilitate the production of subject polymeric matrices that are generally spherical in shape. Substances such as zein, microcrystalline cellulose or microcrystalline cellulose co-processed with sodium carboxymethyl cellulose may confer plasticity to the subject compositions as well as implant strength and integrity. In particular embodiments, during spheronization, extrudates that are rigid, but not plastic, result in the formation of dumbbell shaped implants and/or a high proportion of fines, and extrudates that are plastic, but not rigid, tend to agglomerate and form excessively large implants. In such embodiments, a balance between rigidity and plasticity is desirable. The percent of spheronization enhancer in a formulation depends on the other excipient characteristics and is typically in the range of 10-90% (w/w).

Buffers, acids and bases may be incorporated in the subject compositions to adjust their pH. Agents to increase the diffusion distance of agents released from the polymer matrix may also be included.

Disintegrants are substances which, in the presence of liquid, promote the disruption of the subject compositions. Disintegrants are most often used in implants, in which the function of the disintegrant is to counteract or neutralize the effect of any binding materials used in the subject formulation. In general, the mechanism of disintegration involves moisture absorption and swelling by an insoluble material. Examples of disintegrants include croscarmellose sodium and crospovidone that, in certain embodiments, may be incorporated into the polymeric matrices in the range of about 1-20% of total matrix weight. In other cases, soluble fillers such as sugars (mannitol and lactose) also be added to facilitate disintegration of the subject compositions upon use.

Other materials may be used to advantage to control the desired release rate of a therapeutic agent for a particular treatment protocol. For example, if the sustained release is too slow for a particular application, a pore-forming agent may be added to generate additional pores in the matrix. Any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the polymer system.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores may be varied over a wide range by changing the molecular weight and percentage of pore-forming agent incorporated into the polymer system.

The charge, lipophilicity or hydrophilicity of any subject polymeric matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders. Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices. Three exemplary types of coatings are seal, gloss and enteric coatings. Other types of coatings having various dissolution or erosion properties may be used to further modify subject matrices behavior, and such coatings are readily known to one of ordinary skill in the art.

The seal coat may prevent excess moisture uptake by the matrices during the application of aqueous based enteric coatings. The gloss coat generally improves the handling of the finished matrices. Water-soluble materials such as hydroxypropyl cellulose may be used to seal coat and gloss coat implants. The seal coat and gloss coat are generally sprayed onto the matrices until an increase in weight between about 0.5% and about 5%, often about 1% for a seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as EUDRAGIT, RohmTech, Inc., Malden, Mass., and AQUATERIC, FMC Corp., Philadelphia, Pa., may be used and are layered as thin membranes onto the implants from aqueous solution or suspension or by a spray drying method. The enteric coat is generally sprayed to a weight increase of about one to about 30%, preferably about 10 to about 15% and may contain coating adjuvants such as plasticizers, surfactants, separating agents that reduce the tackiness of the implants during coating, and coating permeability adjusters.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

D. Physical Structures of the Subject Compositions

The subject polymers may be formed in a variety of shapes. For example, in certain embodiments, subject polymer matrices may be presented in the form of microparticles or nanoparticles. Such particles may be prepared by a variety of methods known in the ast, including for example, solvent evaporation, spray-drying or double emulsion methods.

The shape of microparticles and nanoparticles may be determined by scanning electron microscopy. Spherically shaped nanoparticles are used in certain embodiments for circulation through the bloodstream. If desired, the particles may be fabricated using known techniques into other shapes that are more useful for a specific application.

In addition to intracellular delivery of a therapeutic agent, it also possible that particles of the subject compositions, such as microparticles or nanoparticles, may undergo endocytosis, thereby obtaining access to the cell. The frequency of such an endocytosis process will likely depend on the size of any particle.

In certain embodiments, solid articles useful in defining shape and providing rigidity and structural strength to the polymeric matrices may be used. For example, an polymer may be formed on a mesh or other weave for implantation.

The mechanical properties of the polymer may be important for the processability of making molded or pressed articles for implantation. For example, the glass transition temperature may vary widely but must be sufficiently lower than the temperature of decomposition to accommodate conventional fabrication techniques, such as compression molding, extrusion or injection molding.

E. Biodegradability and Release Characteristics

In certain embodiments, the polymers and blends of the present invention, upon contact with body fluids, undergo gradual degradation. The life of a biodegradable polymer in vivo depends, among other things, upon its molecular weight, crystallinity, biostability, and the degree of crosslinking. In general, the greater the molecular weight, the higher the degree of crystallinity, and the greater the biostability, the slower biodegradation will be.

If a subject polymer matrix is formulated with an antineoplastic taxane or other material, release of such a taxene or other material for a sustained or extended period as compared to the release from an isotonic saline solution generally results. Such release profile may result in prolonged delivery (over, say 1 to about 4,000 hours, or alternatively about 4 to about 1500 hours) of effective amounts (e.g., about 0.00001 mg/kg/hour to about 10 mg/kg/hour) of the antineoplastic taxane or any other material associated with the polymer.

A variety of factors may affect the desired rate of hydrolysis of polymers of the subject invention, the desired softness and flexibility of the resulting solid matrix, rate and extent of bioactive material release. Some of such factors include: the selection of the various substituent groups, such as the phosphate group making up the linkage in the polymer backbone (or analogs thereof), the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted. In another example, it is expected that use of substituent on phosphate in the polymers of the present invention that is lipophilic, hydrophobic or bulky group would slow the rate of degradation. For example, it is expected that conversion of the phosphate side chain to a more lipophilic, more hydrophobic or more sterically bulky group would slow down the rate of biodegradation. Thus, release is usually faster from polymer compositions with a small aliphatic group side chain than with a bulky aromatic side chain.

One protocol generally accepted in the field that may be used to determine the release rate of any therapeutic agent or other material loaded in the polymer matrices of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different polymer systems of the present invention may be compared by subjecting them to such a protocol. In certain instances, it may be necessary to process polymeric systems in the same fashion to allow direct and relatively accurate comparisons of different systems to be made. For example, the present invention teaches several different means of formulating the polymeric matrices of the present invention. Such comparisons may indicate that any one polymeric system releases incorporated material at a rate from about 2 or less to about 1000 or more times faster than another polymeric system. Alternatively, a comparison may reveal a rate difference of about 3, 5, 7, 10, 25, 50, 100, 250, 500 or 750. Even higher rate differences are contemplated by the present invention and release rate protocols.

In certain embodiments, when formulated in a certain manner, the release rate for polymer systems of the present invention may present as mono- or bi-phasic. Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 5000 or more ng/day.mg. Alternatively, the release rate may be about 10, 25, 50, 75, 100, 125, 150, 175, 200, 250, 300, 350, 400, 450, 500, 600, 700, 800 or 900 ng/day.mg. In still other embodiments, the release rate of any incorporated material may be 10,000 ng/day.mg or even higher. In certain instances, materials incorporated and characterized by such release rate protocols may include therapeutic agents, fillers, and other substances.

In another aspect, the rate of release of any material from any polymer matrix of the present invention may be presented as the half-life of such material in the such matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

F. Implants and Delivery Systems

In its simplest form, a biodegradable delivery system for an antineoplastic taxane consists of a dispersion of such a therapeutic agent in a polymer matrix. In other embodiments, an article is used for implantation, injection, or otherwise placed totally or partially within the body, the article comprising the subject compositions. It is particularly important that such an article result in minimal tissue irritation when implanted or injected into vasculated tissue.

Biodegradable delivery systems, and articles thereof, may be prepared in a variety of ways known in the art. The subject polymer may be melt-processed using conventional extrusion or injection molding techniques, or these products may be prepared by dissolving in an appropriate solvent, followed by formation of the device, and subsequent removal of the solvent by evaporation or extraction.

Once a system or implant article is in place, it should remain in at least partial contact with a biological fluid, such as blood, internal organ secretions, mucus membranes, cerebrospinal fluid, and the like to allow for sustained release of any encapsulated therpeutic agent, e.g., an antineoplastic taxane.

4. Exemplary Methods of Making the Subject Compositions

In general, the polymers of the present invention may be prepared by melt polycondensation, solution polymerization or interfacial polycondensation. Techniques necessary to prepare the subject polymers are known in the art, and reference is made in particular to U.S. Provisional Application Serial No. 60/216,462 filed Jul. 6, 2000 and U.S. Provisional Application Serial No. 60/228,729 filed Aug. 29, 2000. The most common general reaction in preparing the subject compositions is a dehydrochlorination between a phosphodichloridate and a diol according to the following equation:

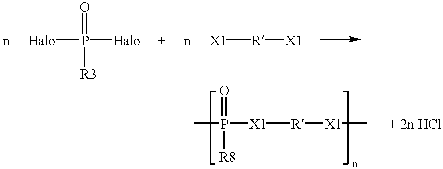

Certain of the subject polymers may be obtained by condensation between appropriately substituted dichlorides and diols.

An advantage of melt polycondensation is that it avoids the use of solvents and large amounts of other additives, thus making purification more straightforward. This method may also provide polymers of reasonably high molecular weight. Somewhat rigorous conditions, however, are often required and may lead to chain acidolysis (or hydrolysis if water is present). Unwanted, thermally induced side reactions, such as cross-linking reactions, may also occur if the polymer backbone is susceptible to hydrogen atom abstraction or oxidation with subsequent macroradical recombination.

To minimize these side reactions, the polymerization may also be carried out in solution. Solution polycondensation requires that both the prepolymer and the phosphorus component be sufficiently soluble in a common solvent. Typically, a chlorinated organic solvent is used, such as chloroform, dichloromethane or dichloroethane. The solution polymerization is generally run in the presence of equimolar amounts of the reactants and, preferably, an excess of an acid acceptor and a catalyst, such as 4-dimethylaminopyridine (DMAP). Useful acid acceptors include tertiary amines as pyridine or triethylamine. The product is then typically isolated from the solution by precipitation in a non-solvent and purified to remove the hydrochloride salt by conventional techniques known to those of ordinary skill in the art, such as by washing with an aqueous acidic solution, e.g., dilute HCl.

Reaction times tend to be longer with solution polymerization than with melt polymerization. However, because overall milder reaction conditions may be used, side reactions are minimized, and more sensitive functional groups may be incorporated into the polymer. The disadvantages of solution polymerization are that removal of solvents may be difficult.

Interfacial polycondensation may be used when high molecular-weight polymers are desired at high reaction rates. By such methods, mild conditions minimize side reactions, and the dependence of high molecular weight on stoichiometric equivalence between diol and dichloridate inherent in solution methods is removed. However, hydrolysis of the acid chloride may occur in the alkaline aqueous phase, and sensitive dichloridates that have some solubility in water are generally subject to hydrolysis rather than polymerization. Phase transfer catalysts, such as crown ethers or tertiary ammonium chloride, may be used to bring the ionized diol to the interface to facilitate the polycondensation reaction. The yield and molecular weight of the resulting polymer after interfacial polycondensation are affected by reaction time, molar ratio of the monomers, volume ratio of the immiscible solvents, the type of acid acceptor, and the type and concentration of the chase transfer catalyst.

Methods for making the present invention may take place at widely varying temperatures, depending upon whether a solvent is used and, if so, which one; the molecular weight desired; the susceptibility of the reactants to form side reactions; and the presence of a catalyst. Usually, the process takes place at a temperature ranging from about 0 to about +235° C. for melt conditions. Somewhat lower temperatures, e.g., for example from about −50 to about 100° C., may be possible with solution polymerization or interfacial polycondensation with the use of either a cationic or anionic catalyst.

The time required for the process may vary widely, depending on the type of reaction being used, the molecular weight desired and, in general, the need to use more or less rigorous conditions for the reaction to proceed to the desired degree of completion. Typically, however, the synthetic process takes place during a time between about 30 minutes and about 7 days.

Although the process may be in bulk, in solution, by interfacial polycondensation, or any other convenient method of polymerization, in many instant embodiments, the process takes place under solution conditions. Particularly useful solvents include methylene chloride, chloroform, tetrahydrofuran, di-methyl formamide, dimethyl sulfoxide or any of a wide variety of inert organic solvents.

In greater detail, polymers of Formula VI may be prepared, at least in part, by reacting a compound having a formula H—Y1—L1—Y1—H, such as 2-aminoethanol, ethylene glycol, ethane dithiol, etc., with a cyclic compound, e.g., having one of the following structures: for example, caprolactone or lactide (lactic acid dimer).

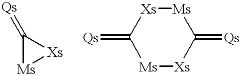

Thus, the cyclic compound may include one or two subunits ts. For cyclic compounds containing two subunits, the two subunits contained therein may be the same or different.

For synthesizing, for example, a compound of Formula VI, wherein x and y are on average about 10, an equivalent of ethylene glycol as H—Y1—L1—Y1—H may be reacted with 20 equivalents of

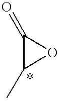

or 10 equivalents of

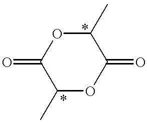

because lactic acid dimer contains two monomer units for each equivalent of the cyclic compound. Variation of the ratio of cyclic compound to ethylene glycol or other bifunctional core will likewise vary the values of x and y, although x and y will be substantially equal for a symmetrical bifunctional core (e.g., ethylene glycol) for subject polymers prepared by this method. For an unsymmetrical bifunctional core (e.g., 2-aminoethanol), the ratio of x:y may vary considerably, as will be understood by one of skill in the art and may be determined without undue experimentation.

Polymers of the present invention may generally be isolated from the reaction mixture by conventional techniques, such as by precipitating out, extraction with an immiscible solvent, evaporation, filtration, crystallization and the like. Typically, the subject polymers are both isolated and purified by quenching a solution of polymer with a non-solvent or a partial solvent, such as diethyl ether or petroleum ether.

5. Dosages and Formulations of the Subject Compositions

In most embodiments, the subject polymers will incorporate the substance to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound from the matrix. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

For the antineoplastic taxanes, a range of dosage is contemplated by the present invention. For infusion of paclitaxel, 135 to 175 mg/M² over about 3 or about 24 hours every three weeks is presently commonly used for treatment of breast carcinoma, lung carcinoma, AIDS-related Kaposi's sarcoma, ovarian carcinoma and other cell proliferative diseases, with or without other theapeutic agents. The present invention contemplates embodiments that release at least those amounts over a three week period, at least twice those amounts over a six week period, etc. Alternatively, the subject compositions may require lower doses than are commonly used today because of their sustained release characteristics.

In other embodiments, the dosage of the subject invention may be determined by reference to the plasma concentrations of the antineoplastic taxane. For example, the maximum plasma concentration (Cmax) and the area under the plasma concentration-time curve from time 0 to infinity (AUC (0-∞)) may be used. For the presently accepted treatments referenced above, it has been reported that Cmax varies from 195 to 3650, and AUC (0-∞) varies from 6300 to 15007. Dosages for the present invention include those that produce the above values for Cmax and AUC (0-∞) and other dosages resulting in larger or smaller values for those parameters.

Alternatively, dosage may be based on the amount of the antineoplastic taxane encapsulated in the subject polymers. For example, a range of amounts of antineoplastic taxanes are contemplated, including 0.5, 1, 2, 3, 4, 5, 7.5, 10, 15, 20, 25 mg or more of such taxanes per kg body weight of the patient. Other amounts will be known to those of skill in the art and readily determined.

The polymers of the present invention may be administered by various means, depending on its intended use, as is well known in the art. For example, if polymer matrices of the present invention are to be administered orally, it may be formulated as tablets, capsules, granules, powders or syrups. Alternatively, formulations of the present invention may be administered parenterally as injections (intravenous, intramuscular or subcutaneous), drop infusion preparations, or suppositories. For application by the ophthalmic mucous membrane route, polymer matrices of the present invention may be formulated as eyedrops or eye ointments. These formulations may be prepared by conventional means, and, if desired, the polymer matrices may be mixed with any conventional additive, such as an excipient, a binder, a disintegrating agent, a lubricant, a corrigent, a solubilizing agent, a suspension aid, an emulsifying agent or a coating agent. In addition, in certain embodiments, polymer matrices of the present invention may be lyophilized or subjected to another appropriate drying technique such as spray drying.

The subject polymer matrices may be administered once, or may be divided into a number of smaller doses to be administered at varying intervals of time, depending in part on the release rate of the matrices and the desired dosage.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of polymer matrix which may be combined with a carrier material to produce a single dose vary depending upon the subject being treated, and the particular mode of administration.

Methods of preparing these formulations or compositions include the step of bringing into association polymer matrices of the present invention with the carrier and, optionally, one or more accessory ingredients. In general, the formulations are prepared by uniformly and intimately bringing into association a subject composition with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition as an active ingredient. Polymer matrices of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the polymer matrices, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject polymer matrices, may contain suspending agents as, for example, ethoxylated isostearyl alcohols, polyoxyethylene sorbitol and sorbitan esters, microcrystalline cellulose, aluminum metabydroxide, bentonite, agar-agar and tragacanth, and mixtures thereof.

Formulations for rectal or vaginal administration may be presented as a suppository, which may be prepared by mixing one or more component with one or more suitable non-irritating excipients or carriers comprising, for example, cocoa butter, polyethylene glycol, a suppository wax or a salicylate, and which is solid at room temperature, but liquid at body temperature and, therefore, will melt in the apropriate body cavity and release the encapsulated bioactive agent.

Formulations which are suitable for vaginal administration also include pessaries, tampons, creams, gels, pastes, foams or spray formulations containing such carriers as are known in the art to be appropriate.

Dosage forms for transdermal administration of includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. For transdermal administration, the complexes may include lipophilic and hydrophilic groups to achieve the desired water solubility and transport properties.

The ointments, pastes, creams and gels may contain, in addition to subject polymeric matrices, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Polymeric matrices may alternatively be administered by aerosol. This is accomplished by preparing an aqueous aerosol, liposomal preparation or solid particles. A non-aqueous (e.g., fluorocarbon propellant) suspension may be used. Sonic nebulizers may be used because they minimize exposing the agent to shear, which may result in degradation of the compound. Ordinarily, an aqueous aerosol is made by formulating an aqueous solution or suspension of the polymeric materials together with conventional pharmaceutically acceptable carriers and stabilizers. The carriers and stabilizers vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Ophthalmic formulations, eye ointments, powders, solutions and the like, are also contemplated as being within the scope of this invention.

Certain pharmaceutical compositions of this invention suitable for parenteral administration comprise one or more polymeric materials in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

In certain embodiments, the subject compositions comprise about 5% to about 60%, alternatively about 10% to about 50% of an antineoplastic taxane, such as paclitaxel or docetaxel, in a biodegradable polymer, such as a phosphorous-based polymer, e.g., D,L-PL(PG)EOP, described in the Exemplification section below. In certain embodiments, a composition comprises at least about 10% of an antineoplastic taxane, more particularly at least about 20%, or even more than about 30% of an antineoplastic taxane, such as paclitaxel or docetaxel. In certain embodiments, the compositions are formulated as microparticles, microspheres or nanospheres. The compositions may additionally comprise cholesterol or another suitable excipient that improves the physical characteristics, such as flowability, viscosity, glass temperature etc., of the subject composition for the particular use. Microsphere compositions may be suspended in a pharmaceutically acceptable solution, such as saline, Ringer's solution, dextran solution, dextrose solution, sorbitol solution, a solution containing polyvinyl alcohol (from about 1% to about 3%, preferably about 2%), or an osmotically balanced solution comprising a surfactant (such as Tween 80 or Tween 20) and a viscosity-enhancing agent (such as gelatin, alginate, sodium carboxymethylcellulose, etc.). In certain embodiments, the composition is administered subcutaneously. In other embodiments, the composition is administered intravenously. For intravenous delivery, the composition is preferably formulated as microspheres on average less than about 20 microns, more particularly less than about 15 microns, and still more particularly less than about 10 microns in average diameter.

### EXEMPLIFICATION

The invention now being generally described, it will be more readily understood by reference to the following examples which are included merely for purposes of illustration of certain aspects and embodiments of the present invention, and are not intended to limit the invention.

### EXAMPLE 1

### First Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of 1,2-propanediol (PG), obtained from Aldrich, Catalog No. 39,803-9, 99.5+ %, in a molar ratio of 10:1, were weighed into a 250 mL 3-neck round-bottom flask. The flask was equipped with a gas joint and a stirrer bearing/shaft/paddle assembly. The mixture was evacuated and pressurized with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene of chloroform) equivalent to 3.6 mg tin (120 ppm stannous octoate or equivalent to 35 ppm tin based upon weight of the prepolymer) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours. A reflux condenser was then inserted between the gas joint and the flask in the prepolymer apparatus described above. The molten prepolymer was dissolved by adding 100 mL of chloroform to the reaction flask with stirring.

Next, 6.9 mL of triethylamine (TEA) and 1.21 g of DMAP were added to the stirring reaction mixture. The reaction mixture was then chilled to about 4° C. in an ice bath. A solution of approximately 2.5 mL of freshly distilled ethyl dichlorophosphate (EOPCl₂) in 25 mL of chloroform was prepared in a dropping funnel. The solution in the funnel was added drop wise to the reaction mixture over a period of about 30 minutes. After the addition was complete the reaction mixture was allowed to continue stirring at about 4° C. for 10 minutes and then the ice bath was removed. The reaction mixture was allowed to warm to room temperature over about 1 hour. At this time a significant increase in viscosity of the clear solution was observed. The reaction mixture was then heated to reflux using an oil bath. Over the next hour the solution became cloudy. The reaction mixture was allowed to reflux over two nights, about 38 hours total.

At this time, a Barret trap was inserted between the condenser and the flask and 88 mL of solvent (⅔ of the total volume) were distilled from the reaction mixture. The Barret trap was removed and the reaction mixture was allowed to reflux for an additional 16 hours with the oil bath temperature between 98-102° C. Next, the oil bath temperature was increased to 115° C. for 2 hours. After this time, the reaction mixture was allowed to cool to room temperature, and 200 mL of dichloromethane was added and transferred to a separatory funnel. The reaction mixture was extracted twice with 100 mL of 0.1 M HCl and twice with 100 mL of saturated sodium chloride solution. The organic layer was isolated, dried overnight in the freezer at about −15° C. over 50 g of sodium sulfate, and filtered twice. The resulting polymer solution was poured into 1500 mL of hexane plus 500 mL of ether. The resulting mass of polymer was dried under vacuum. The Inherent Viscosity (IV) of this material was measured to be 0.39 dL/g.

### EXAMPLE 2

### Second Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. Each time the polymerization vessel was evacuated to a pressure between 0.5 and 10 Torr. The reaction apparatus was immersed in a preheated oil bath at 125° C., connected to an argon source with an oil bubbler, and stirred at a moderate speed until all of the solid monomer had melted. At this time, a volume of stock stannous octoate solution (about 130 mg/ml in toluene) equivalent to 100 ppm stannous octoate (29 ppm Sn) was added to the melt using a syringe. The reaction mixture was allowed to stir under a slight argon pressure for 3 hours. The oil bath temperature was then reduced to about 105° C. and the residual monomer was removed under vacuum. The pressure was maintained as low as possible, typically between 0.5 and 10 Torr. The upper parts of the reaction assembly were heated gently with a heat gun to aid in the monomer removal. The total time under vacuum was 1 hour.

The prepolymer was cooled to room temperature under argon gas and allowed to stand for 12-18 hours at ambient temperature. The prepolymer was dissolved in 84 ml of chloroform with stirring and 2.5 equivalents of triethylamine (TEA) and 0.5 equivalents of DMAP were added to the stirring reaction mixture using a powder funnel. The reaction mixture was chilled to about −5 to about −15° C. in a cold bath. A solution of about 1 equivalent of distilled ethyl dichlorophosphate (EOPCl₂) in 10 ml of chloroform was prepared in a dropping funnel. The solution in the funnel was added slowly to the reaction mixture over a period of 0.5 hour.

After the addition was complete, the reaction mixture was allowed to stir at low temperature for 1 hour at −5° C. The reaction was then quenched with 1 ml of anhydrous methanol and stirred for another five minutes. Next, the reaction mixture was transferred to a 0.5 gallon vessel and mixed with 37 g of Dowex DR-2030 IER and 30 g of Dowex M-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper.

The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. Molecular weights of the polymers were obtained from gel permeation chromatography (GPC) using both differential refractive index detection and a polystyrene calibration curve (CC) and by light scattering detection. The molecular weight and IV data for the polymers prepared by this process are listed in the table below.

| Sample | Mw (LS), daltons | Mw (CC), daltons | IV, dL/g |
|---|---|---|---|
| 1 | 101,200 | 107,500 | 0.62 |
| 2 | 150,100 | 155,900 | 0.80 |
| 3 | 85,200 | 84,300 | — |
| 4 | 92,600 | 89,900 | — |

### EXAMPLE 3

### Synthesis of D,L-PL(EG)EOP

All glassware was dried for a minimum of 2 hours at 105° C. and allowed to cool in a desiccator or cooled under a stream of argon gas. A 100.0 g portion of D,L-lactide and 4.3 g of ethylene glycol (EG) (molar ratio, 10: 1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 40,400 for Mw (LS) and 42,000 for Mw (CC).

### EXAMPLE 4

### Synthesis of D,L-PL(HD)EOP

All glassware was dried for a minimum of 2 hours at 105° C. and allowed to cool in a desiccator or cooled under a stream of argon gas. A 100.0 g portion of D,L-lactide and 8.2 g of 1,6-hexane diol (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution equivalent (about 130 mg/ml in toluene) to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of triethylamine (TEA) and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours, a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 36,700 for Mw (LS) and 34,100 for Mw (CC). The value for IV was 0.33 dL/g.

### EXAMPLE 5

### Polymer of PG, D,L-lactide, Glycolide and Ethyl Dichlorophosphate

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly and a 125 ml dropping funnel containing 4.6 g of glycolide. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. At this time the glycolide was melted using a heat gun and added to the polymer melt in the flask. The melt was stirred for an additional 2 hours. The oil bath temperature was then reduced to about 115° C. and the residual monomer was removed under vacuum. The upper parts of the reaction assembly were heated gently with a heat gun to aid in the monomer removal. The total time under vacuum was 2 hours.

The molten prepolymer was suspended in 84 ml of chloroform with stirring and 2.5 equivalents of TEA and 0.5 equivalents of DMAP were added to the stirring reaction mixture using a powder funnel. The reaction mixture was chilled to about 4° C. in a cold bath. A solution of about 1 equivalent of distilled ethyl dichlorophosphate (EOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another 1.75 hours and then the cold bath was removed. The reaction mixture was allowed to warm to room temperature and stirred for 2 to 18 hours. After 2 hours a significant increase in viscosity of the clear solution was observed. The reaction was then quenched with 1 ml of anhydrous methanol and stirred for another five minutes.

Next, 37 g of dry Dowex HCR-S IER and 30 g of dry Dowex M-43 were added to the reaction mixture and stirring was continued for another hour to remove residual DMAP and TEA free base and salts. The IERs were removed from the reaction mixture by vacuum filtration through Whatman 54 filter paper. The resin was washed with about one bed volume of dichloromethane and the filtrate was concentrated to approximately 50 ml. The viscous filtrate was poured into 700 ml of petroleum ether to precipitate the polymer and dried under vacuum.

### EXAMPLE 6

### Synthesis of D,L-PL(PG)HOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10: 1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 100 ml of chloroform with stirring and TEA and DMAP were added to the stirring reaction mixture using a powder funnel. The funnel was rinsed with 10 ml of chloroform. The reaction mixture was chilled to about 4° C. in a cold bath. A solution of about 1 equivalent of distilled hexyl dichlorophosphate (HOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another hour and then the cold bath was removed. The reaction mixture was allowed to warm to room temperature and stirred for 2 to 18 hours. After 2 hours a significant increase in viscosity of the clear solution was observed. The reaction was then quenched with 800 μl of anhydrous methanol and stirred for another five minutes.

Next, Dowex MR-3C ion exchange resin (IER) was added to the reaction mixture and stirring was continued for another hour to remove residual DMAP and TEA free base and salts (the Dowex resin had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 100 ml. The viscous filtrate (now a somewhat cloudy solution) was poured into 1000 ml of hexane to precipitate the polymer. The polymer mass was washed with 2×200 ml of hexane and dried under vacuum. The molecular weight and IV data for the polymers prepared by this process are listed in the table below.

| Sample | Mw (LS), daltons | Mw (CC), daltons | IV, dL/g |
|---|---|---|---|
| 1 | 64,200 | 58,000 | 0.48 |
| 2 | 68,000 | 62,700 | 0.43 |

### EXAMPLE 7

### Synthesis of D,L-PL(PG)EP

All glassware was dried for a minimum of 2 hours at 105° C. and allowed to cool in a desiccator or cooled under a stream of argon gas. A 28.5 g portion of D,L-lactide and 1.5 g of PG (molar ratio, 10: 1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 130° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for 4 hours. The oil bath temperature was then reduced to about 110° C. and the residual monomer was removed under vacuum. The upper parts of the reaction assembly were heated gently with a heat gun to aid in the monomer removal. The total time under vacuum was 2 hours.

The molten prepolymer was dissolved in 84 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphonate (EPCl₂) in 9 ml of chloroform was prepared in a dropping funnel. The solution in the funnel was added slowly to the reaction mixture over a period of 0.5 hour. After the addition was complete, the viscosity of the solution had increased significantly and the reaction mixture was allowed to stir at low temperature for 1 hour at −5° C. The reaction was then quenched with 1 ml of anhydrous methanol and stirred for another five minutes.

Next, the reaction mixture was transferred to a 0.5 gallon vessel and mixed with 37 g of Dowex DR-2030 IER and 30 g of Dowex-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. The molecular weight data for the polymers prepared by this process are listed in the table below.

| Sample | Mw (LS), daltons | Mw (CC), Daltons |
|---|---|---|
| 1 | 339,900 | 327,600 |
| 2 | 369,800 | 360,900 |

### EXAMPLE 8

### Synthesis of P(cis- and trans-CHDM/HOP)

All glassware was dried for a minimum of two hours at 105° C. and allowed to cool in a desiccator or cooled under a stream of argon gas. A reaction assembly consisting of a 1 L three-neck round-bottom flask equipped with a gas joint, a stirrer bearing/shaft/paddle and a dropping funnel. A solution of 20.0 g of 1,4-cyclohexane dimethanol (CHDM) was prepared in 75 ml of anhydrous tetrahydrofuran (THF) and transferred to the reaction vessel. The beaker was rinsed with 25 ml of THF and the wash was transferred to the reaction vessel.

Next, 29.0 ml of N-methylmorpholine (NMM) and 1.61 g of DMAP were added to the reaction mixture through a powder funnel. A solution of 28.86 g of hexyl dichlorophosphate (HOPCl₂) in 30 ml of THF was prepared under argon and transferred to the dropping funnel while the reaction mixture was cooled to 4° C. in a cold bath. The solution in the funnel was added to the reaction mixture over a period of one hour. With 5 to 10 minutes after the start of addition, a white precipitate, presumably the hydrochloride salts of NMM and DMAP, began to form. After the addition was complete the funnel was rinsed with 30 ml of THF. The reaction mixture was stirred for 1 hour at 4° C. and then for either 2 or 18 hours at ambient temperature.

At the prescribed time, the precipitate was removed from reaction mixture by vacuum filtration. The filtrate was diluted with 100 ml of dichloromethane, transferred to a half-gallon jar and 86.5 of dried Dowex HCR-S IER and 103.8 g of dried Dowex M-43 IER were added to the filtrate. The jar was sealed with a Teflon lined lid and the mixture was agitated on a mechanical shaker for two hours.

At this time, the IERs were removed by vacuum filtration and the filtrate was concentrated to approximately 100 ml under vacuum. The polymer solution was poured in 2 L of hexane and the resulting fluid material that precipitated was isolated and transferred to a Teflon lined glass dish. The polymer was dried under vacuum to yield a sticky, free flowing viscous liquid. The Mw (LS) data for the polymers prepared by this process are listed in the table below.

| Sample | Mw (LS), daltons | Mw (CC), daltons | IV, dL/g |
|---|---|---|---|
| 1 | 4400 | 5500 | 0.14 |
| 2 | 5000 | 6500 | 0.11 |
| 3 | 4000 | 4600 | 0.10 |

### EXAMPLE 9

### Synthesis of P(BHET/EOP)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of bis(hydroxyethyl) terephthalate (BHET) and 28.83 g of DMAP were added to the reaction vessel using a powder funnel and mixed with 81 ml of THF. The solids were dissolved with stirring and gentle heating using a heat gun.

After all solids had dissolved, the reaction mixture was cooled to 4° C. in a cold bath. A solution of 19.2 g of ethyl dichlorophosphate (EOPCl₂) in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. After all of the solution in the funnel had been added, the stirrer shaft/paddle became entrapped in a thick, stiff precipitate and stirring ceased. It appears the polymer that had formed at this time was insoluble in the reaction mixture.

Next, 125 ml of dichloromethane were added and the reaction mixture was swirled by hand until mechanical stirring could be resumed. The reaction mixture was now a homogenous solution containing a white free flowing powder. The reaction mixture was stirred at 4° C. for one hour. The cold bath was removed and the reaction mixture was allowed to warm to ambient temperature and stirred for 16 hours. At this time, the white precipitate was removed from the reaction mixture by vacuum filtration and the filter cake was washed with 100 ml of dichloromethane.

The resulting filtrate was transferred to a half-gallon jar and treated with 156.92 g of undried Dowex HCR-S IER and 160.92 g of undried Dowex M-43 IER. The resins were washed with 2 bed volumes of methanol and 2 bed volumes of dichloromethane prior to use. The jar was sealed with a Teflon lined lid and shaken on a mechanical shaker for two hours. The resin was removed by vacuum filtration and the filtrate, ˜600 ml, was concentrated to ˜150 ml. The clear solution was poured into 1.2 L of hexane. The thick oil that precipitated was washed with 400 ml of hexane and transferred to a Teflon lined glass dish, dried under vacuum. The molecular weights were determined by GPC were 2200 for Mw (LS) and 2100 for Mw (CC). The value obtained for IV was 0.10 dL/g.

### EXAMPLE 10

### Synthesis of P(BHET-EOP/TC)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of BHET and 28.83 g of DMAP were added to the reaction vessel using a powder funnel and mixed with 81 ml of THF and 125 ml of dichloromethane.

The solids were dissolved with stirring and gentle heating using a heat gun. After all solids had dissolved, the reaction mixture was cooled to 4° C. in a cold bath. A solution of 19.2 g of EOPCl₂ in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. The reaction mixture was stirred at 4° C. for one hour. Next, a solution of 4.79 g of terephthaloyl chloride (TC) in 18 ml of THF was prepared in the addition funnel and added to the solution in the flask over a 30-minute period. The reaction mixture was stirred for one hour at 4□C.

At this time the cold bath was removed and the reaction was allowed to warm to room temperature and stir for another 20 hours. At this time, the white precipitate was removed from the reaction mixture by vacuum filtration. The resulting filtrate was transferred to a half-gallon jar and treated with 88.5 g of dried Dowex HCR-S IER and 73.8 g of dried Dowex M-43 IER. The jar was sealed with a Teflon-lined lid and shaken on a mechanical shaker for two hours. The resin was removed by vacuum filtration and the filtrate was concentrated to ˜100 ml. The clear solution was poured into 2 L of hexane. The thick oil that precipitated was transferred to a Teflon-lined glass dish, dried under vacuum. The molecular weights were determined by GPC were 7200 for Mw (LS) and 4000 for Mw (CC). The value obtained for IV was 0.09 dL/g.

### EXAMPLE 11

### In Vivo Release of Paclitaxel from D,L-PL(PG)EOP

The term “PACLIMER” shall refer to a subject polymer in a microsphere form with the D,L-PL(PG)EOP composition containing paclitaxel at certain loading levels. The D,L-PL(PG)EOP polymer in PACLIMER may be prepared using the method described in Example 1 or 2 above or Example 19 below, with Example 2 and 19 now being the preferred method of synthesis. If there is no designation after PACLIMER, then paclitaxel is loaded at the 10% level; otherwise, the loading level will be expressly stated or alternatively indicated in parantheses as shown for the following examples: for 30% loading level, “PACLIMER (30%)”; for fifty percent loading, “PACLIMER (50%)”; etc. All microspheres, unless otherwise indicated, were prepared using the solvent dilution method described below.

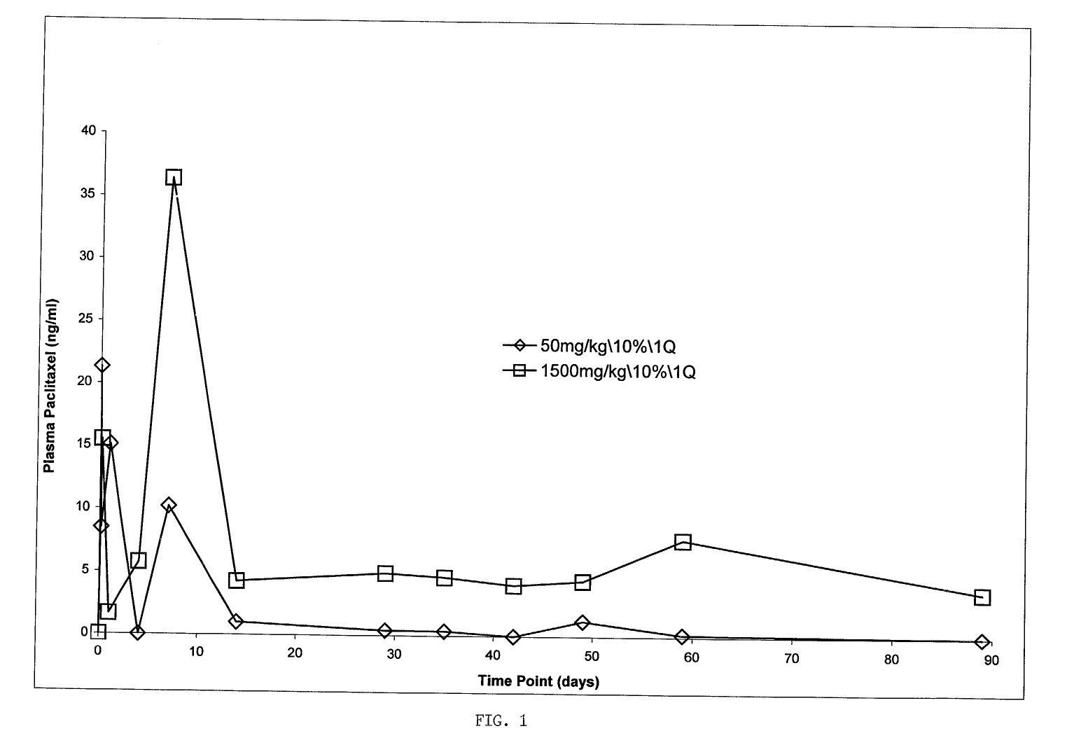

120 female C57BL/6 mice each weighing 15-20 g were provided at the onset of the study. The animals were randomly divided into 12 groups of n=5 for measurement at pre-dose, 4 hour, 1, 7, 14, 28, 35, 42, 49, 60 and 90 days post dose. PACLIMER, dosed at 50 and 1500 mg/kg were administered sub-cutaneously as a suspension in saline/tween vehicle. The dose calculation was based on average weight across all animals in the study. Dose materials were kept refrigerated until use, then warmed to room temperature. Each animal was dosed 0.5 ml into the right flank, via a 3 cc multi-dose syringe. Animals were observed approximately 4 hrs post dose, daily for the first 3 days, then weekly (at sacrifices) throughout the duration of the study. At sacrifice, animals were exsanguinated via dry ice/CO₂. Blood was drawn through a cardiac puncture for centrifugation/recovery of the plasma layer. Plasma was transferred to a clean tube. Samples were frozen at −40° C. prior to in-house analysis by LC/MS/MS. The plasma concentration of paclitaxel was measured; results are presented in FIG. 1. As indicated, administration of PACLIMER at a dose of 1500 mg/kg resulted in a plasma level of paclitaxel above 4 ng/ml being maintained for over 50 days.

### EXAMPLE 12

### In Vivo Efficacy of Microsphere-Encapsulated Paclitaxel

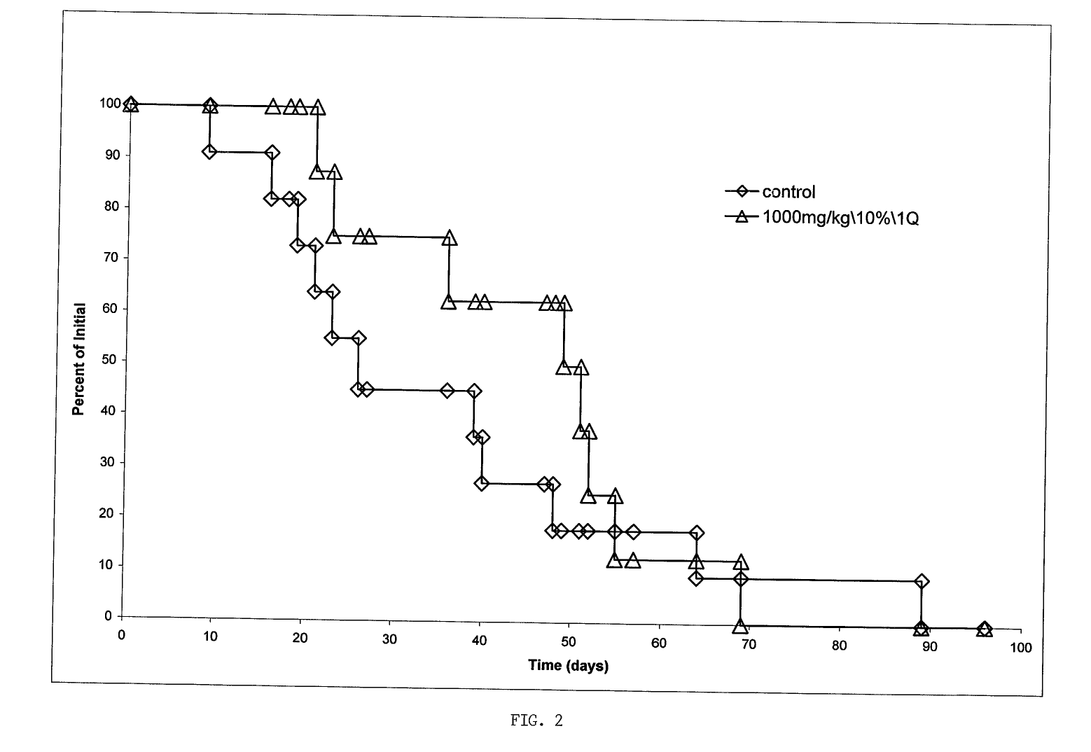

35 female C57BL/6 mice, 5-6 weeks of age at onset of study, were provided, of which 10 were maintained in a control group and 10 treated with 1000 mg/kg PACLIMER, with n=5 sentinels to confirm metastases. Under anesthesia, 5.0×10⁵ Lewis Lung murine carcinoma cells per animal were administered through the tail vein in 0.1 ml of RPMI cell culture media. The animals were randomized into treatment groups at cell inoculation. At 5 days metastases were confirmed in 2 of 2 sentinel animals, and treatments carried out. PACLIMER dosed at 1000 mg/kg were administered subcutaneously suspended in sodium carboxymethylcellulose (NaCMC) vehicle. Dose materials were kept refrigerated until use, then warmed to room temperature. Pre-packaged 1 g PACLIMER was suspended to yield a 100 mg/ml dose article. Dose calculation was based on individual animal weight of each animal in study. An appropriate dose volume was drawn into a 1 cc syringe, then q.s. to 1 ml with normal saline. Each animal was treated with 1.0 ml of this solution into the right flank. Treated animals were observed for duration of life against no treatment controls. Results of this experiment are presented in FIG. 2. Median survival time was approximately doubled in the group receiving a subcutaneous dose of PACLIMER.

### EXAMPLE 13

### Comparison of PACLIMER Delivery Techniques and Doses

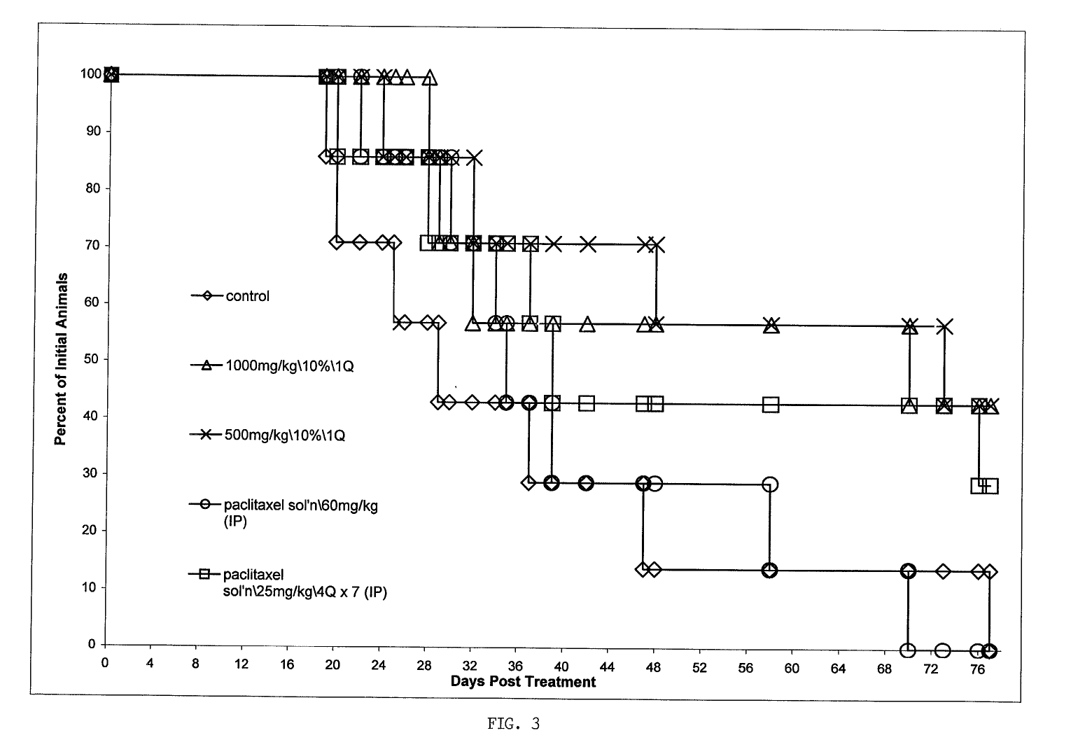

70 female C57BL/6 mice, 5-6 weeks old, were provided (due to losses at anesth., only 49 animals survived to study). They were divided into groups of 7 for control, 500 and 1000 mg/kg PACLIMER, 60 mg/kg dose of paclitaxel sol'n IP (intraperitoneal) 25 mg/kg IP Taxol commercial preparation 4Q×7 (one treatment every seven days for four weeks), and sentinels to confirm metastases. Under anesthesia, 5.0×10⁵ Lewis Lung murine carcinoma cells per animal were administered through the tail vein in 0.1 ml of RPMI cell culture media. The animals were randomly divided into treatment groups at time of cell inoculation. No metastases were present at 5 days, but were confirmed at 7 days, and treatments were carried out. Dose materials were kept refrigerated until use, then warmed to room temperature. Pre-packaged 1 g PACLIMER was suspended in saline/tween vehicle to yield a 100 mg/ml dose article. Dose calculation was based on individual weight of each animal in the study. Appropriate dose volume was drawn into a 1 cc syringe. Each IP (intraperitoneal) PACLIMER dose was q.s. to 1 ml with normal saline, and given bolus, via a single dose syringe. Each SC (subcutaneous) PACLIMER dose was given into the right flank without further dilution. Paclitaxel sol'n dose was prepared in 1:1 EtOH:cremophor, then diluted 1:10 with normal saline. Each IP paclitaxel dose q.s. to 1 ml with normal saline, and given bolus, via a single dose syringe. The animals were observed for duration of life against no treatment controls. Results of this experiment are presented in FIG. 3. Median survival time was improved by more than a factor of two for the groups treated with subcutaneous adminstration of PACLIMER at either dosage level.

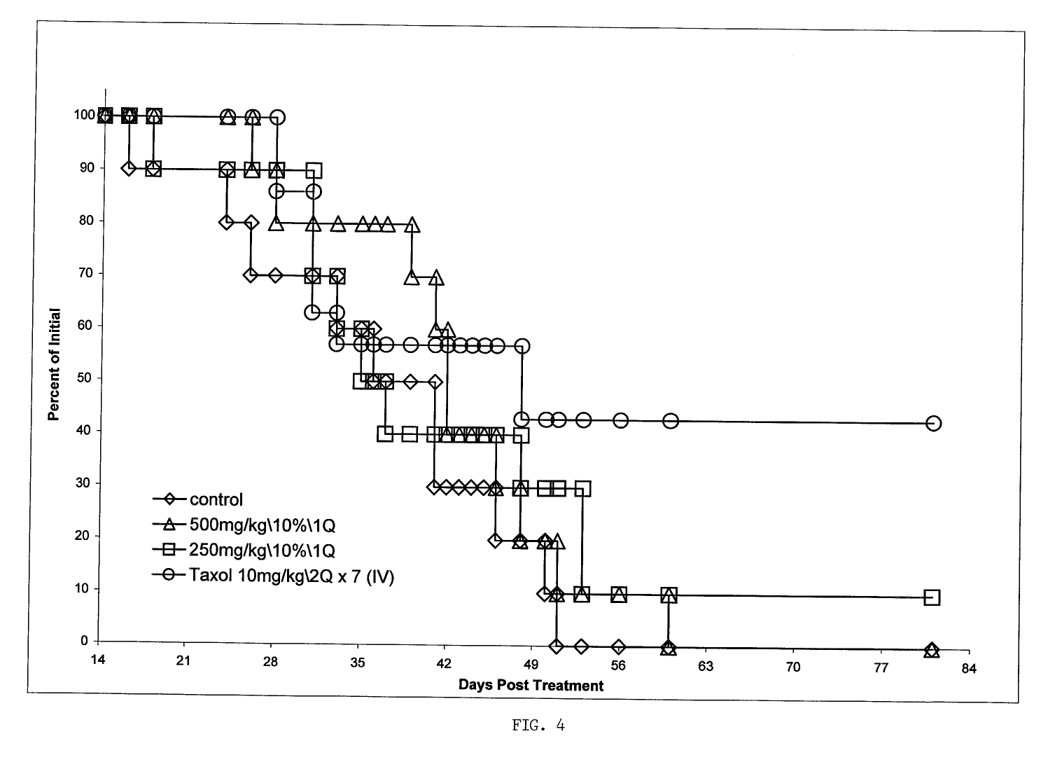

A second experiment was carried out using 70 female C57BL/6 mice, 5-6 weeks old at onset of study, which were divided into groups of 10 for control, 250 & 500 mg/kg PACLIMER (30%) for subcutaneous administration (n=9), 25 mg/kg IV Taxol commercial preparation 2Q×7 and sentinels to confirm metastases. Under anesthesia, 5.0×10⁵ Lewis Lung murine carcinoma cells/animal were administered through the tail vein in 0.1 ml of RPMI cell culture media. The animals were randomly divided into treatment groups at the time of cell inoculation. Metastases were confirmed at 7 days, and treatments were carried out. Dose materials were kept refrigerated until use, then warmed to room temperature. PACLIMER was suspended in saline/tween vehicle to yield one 18 mg/ml dose sol'n for all groups. Dose calculations were based on average animal weight of this strain at this age. Appropriate dose volume was drawn into a 3 cc syringe. Each PACLIMER or microsphere dose was given bolus undiluted, via a multi-dose syringe. Commercial Taxol preparation was given IV, undiluted through the tail vein, via a 1 cc multi-dose syringe. All animals were observed, and clinical signs noted, approximately 4 hrs. post dose, then monitored for duration of life against no treatment controls. Results are presented in FIG. 4.

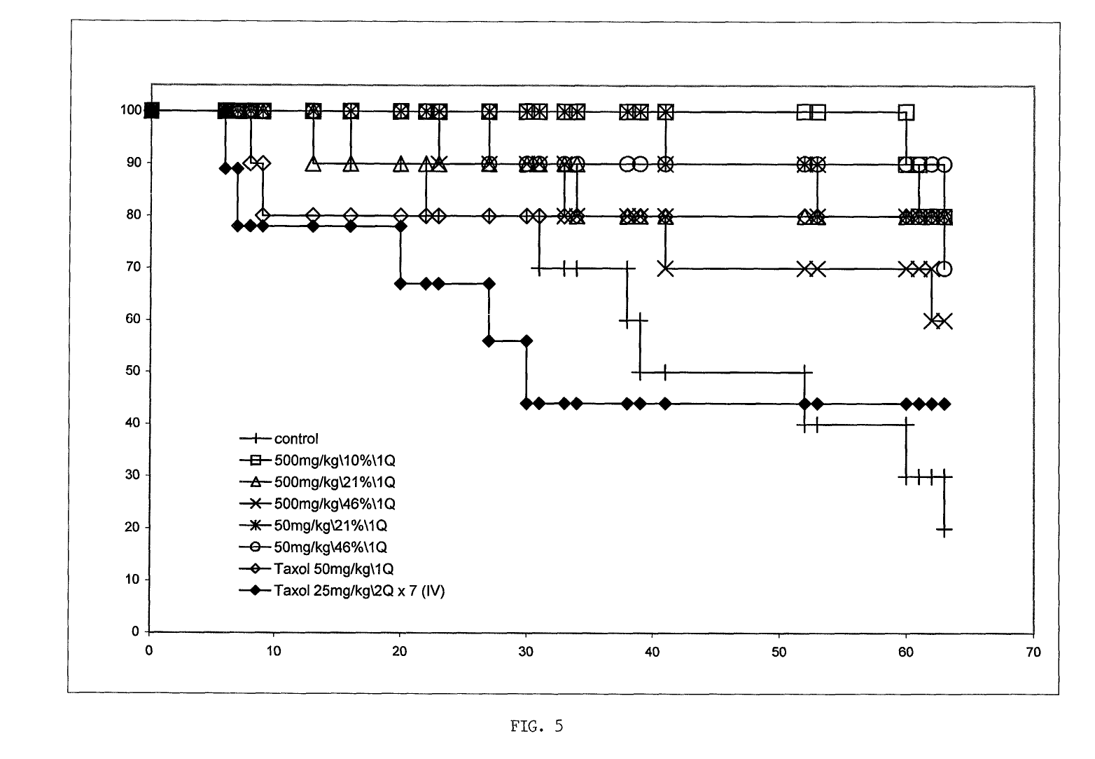

A third experiment was performed using 90 male C57BL/6 mice, 5-6 weeks old at onset of study, divided into groups of 10 for control, 500 mg/kg PACLIMER with 10, 21 and 46% loading of paclitaxel; 50 mg/kg PACLIMER with 21 and 46% paclitaxel loading; 50 mg/kg Taxol; 25 mg/kg Taxol IV 2Q×7 (one of these animals died under anesthesia, and the study was complete with n=9); and sentinels. Under anesthesia, 5.0×10⁵ Lewis Lung murine carcinoma cells per animal were administered through the tail vein in 0.1 ml of RPMI cell culture media. The animals were randomly divided into treatment groups at time of cell inoculation. Metastases were confirmed at 7 days, and treatments carried out. Dose materials were kept refrigerated until use, then warmed to room temperature. Dose calculation was based on average animal weight across a sampling of animals on study. Appropriate PACLIMER dose volume drawn into a 3 cc syringe mulit-dose syringe. All PACLIMER doses were suspended in saline/tween vehicle. Each PACLIMER dose was given as a 0.5 ml bolus into the right flank. Appropriate commercial Taxol preparation volume was drawn into a 1 cc single dose syringe for IV & SC. IV Taxol treatments q.s. to 0.1 ml and delivered bolus through the tail vein. SC Taxol treatments q.s. to 0.5 ml and delivered bolus into the right flank. All animals were observed, and clinical signs noted, approximately 4 hrs. post dose, then monitored for duration of life against no treatment controls. Clinical reactions/changes at the SC injection site were noted. Average group weights were monitored weekly. Results of the survival study are presented in FIG. 5. At the conclusion of the study, groups treated with PACLIMER had a survival rate at least three times that of the control group, and certain treatment groups had a survival rate four times that of the control group.

### EXAMPLE 14

### In Vivo Release of Paclitaxel from IV PACLIMER

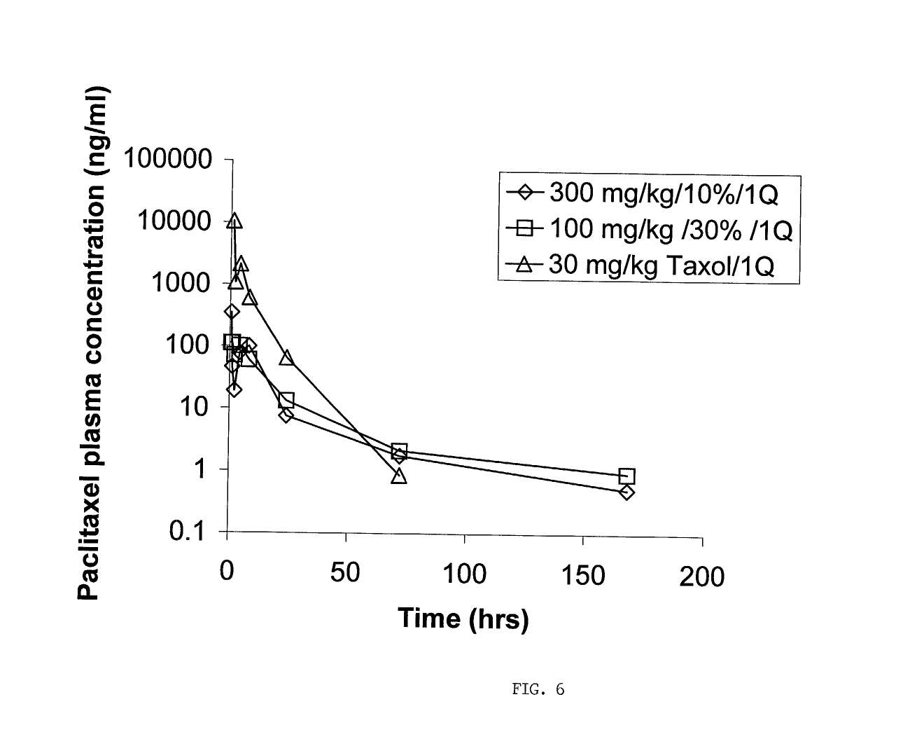

84 female C57BL/6 mice, 6-7 weeks old at onset of study, were provided and randomly divided into groups of three for monitoring at pre-dose, 0.5, 1, 2, 4 and 8 hours, 1, 3, and 7 days post dose for each treatment regimen. One regimen was PACLIMER dosed at 300 mg/kg IV (6.49 um (Mv)), another was 100 mg/kg of PACLIMER (30%) (8.50 um (Mv)), and the third was 30 mg/kg Taxol 1Q. PACLIMER microspheres were suspended in saline/tween vehicle. Dose materials were kept refrigerated until use, then warmed to room temperature. Each animal was dosed 0.1 ml through the tail vein, via a 1 cc multi-dose syringe. The animals were observed approximately 4 hrs post dose, then at sacrifices for the duration of the study. At sacrifice, animals were exsanguinated via dry ice/CO₂. Blood was drawn through a cardiac puncture for centrifugation/recovery of the plasma layer. Plasma was transferred to a clean tube and stored at −40° C. prior to analysis via LC/MS/MS. Results are portrayed in FIG. 6. Plasma concentrations of paclitaxel were maintained above 2 ng/mL only when microspheres were administered. Furthermore, the highest plasma concentration of paclitaxel was less than 250 times greater than the lowest concentration measured over the first 72 hours in the microsphere treatments, whereas administration of Taxol solution IV resulted in a range that varied over a range of 12,000 times the lowest measured concentration. Thus, the plasma concentration of paclitaxel resulting from the microsphere administration was considerably more constant than the concentration resulting from administration of a simple solution of paclitaxel, the range being diminished by a factor of over 50.

### EXAMPLE 15

### In Vivo Release of Paclitaxel from PACLIMER

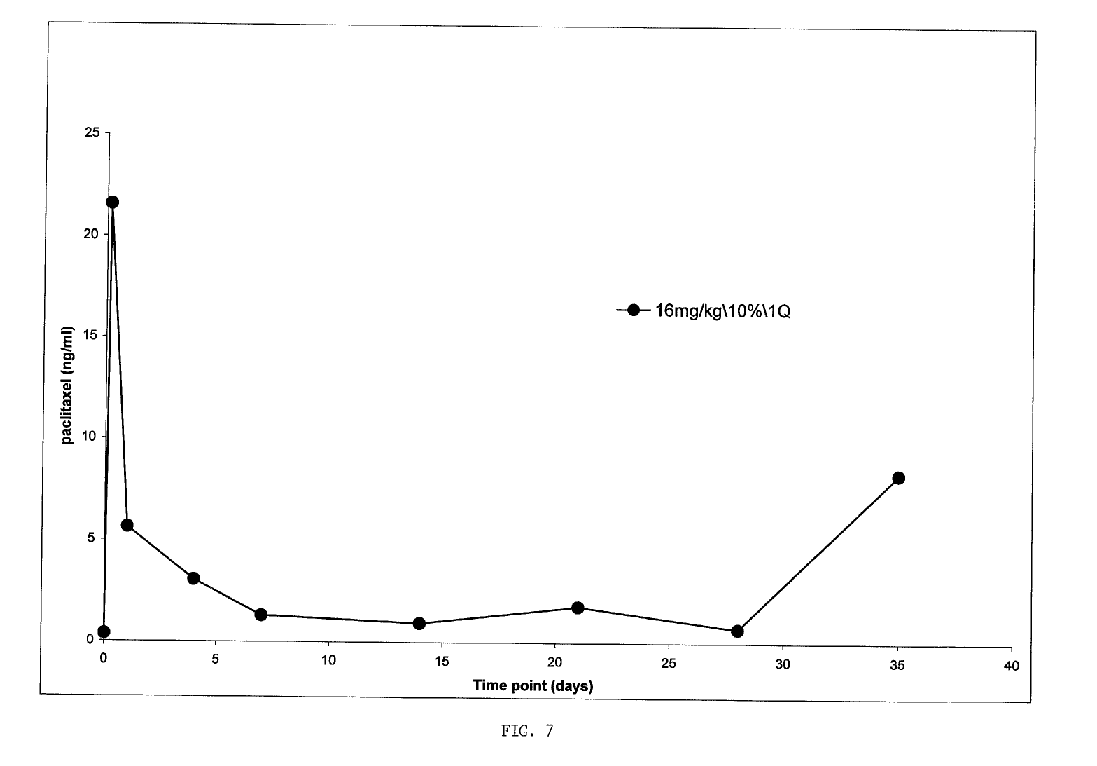

45 female ICR (CD-1) mice, 24-35 g, at onset of study, were randomly divided into 9 groups of n=5 for time points of: pre-dose, 4 hour, 1, 3, 7, 14, 21, 28 and 35 days post dose. PACLIMER microspheres were dosed at 160 mg/kg sub-cutaneously suspended in NaCMC vehicle. Dose calculations were based on average weight across all animals on study. Dose materials were kept refrigerated until use, then warmed to room temperature. Each animal was dosed 0.5 ml into the right flank, via a 3 cc multi-dose syringe. The animals were observed approximately 4 hrs post dose, daily for the first 3 days, then weekly (at sacrifices) throughout the duration of the study. At sacrifice, animals were exsanguinated via dry ice/CO₂. Blood was drawn through cardiac puncture for centrifugation/recovery of the plasma layer. Plasma was transferred to a clean tube and buffered with ½ plasma volume of 1 M sodium acetate buffer (pH=3.9). Samples were frozen at −80° C. until analysis by LC/MS/MS. Results are depicted in FIG. 7. On average, plasma concentrations of paclitaxel were maintained within a factor of 40 over a range of three days.

### EXAMPLE 16

### Survival with IV Administration of PACLIMER

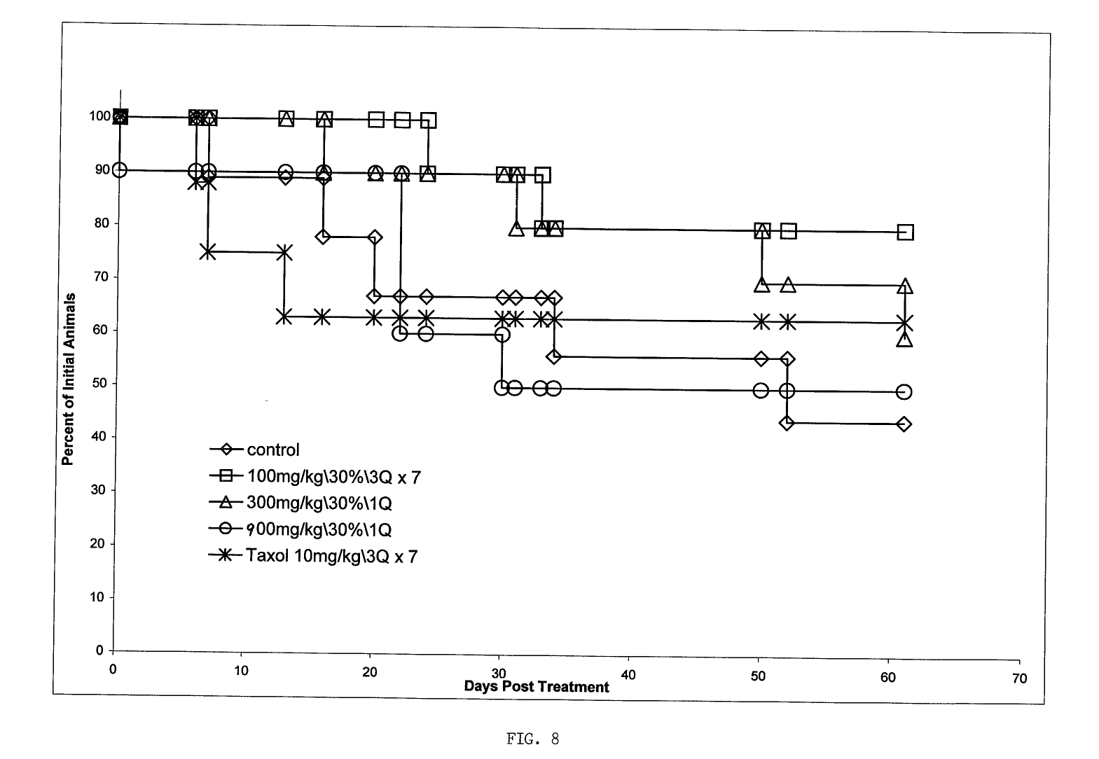

60 female C57BL/6 mice, 5-6 weeks old at study onset, were divided into groups of 10 for control, 300 & 900 mg/kg PACLIMER (30%), 100 mg/kg PACLIMER 3Q×7 (30%) and 30 mg/kg Taxol 3Q×7, with 10 sentinels to confirm metastases. There were two immediate deaths at dosing in the Taxol 10 mg/kg 3Q×7 group. These animals were removed from the data, and all calculations of percent survival for that group are based on n=8. Under anesthesia, 5.0×10⁵ Lewis Lung murine carcinoma cells per animal were inoculated through the tail vein in 0.1 ml of RPMI cell culture media. Animals were randomly divided into treatment groups at cell inoculation. At 7 days, metastases were confirmed in 2 of 2 sentinel animals, and treatments were carried out. PACLIMER (30%) microspheres prepared by solvent dilution were suspended in saline/tween vehicle were dosed IV through the tail vein. Dose materials were kept refrigerated until use, then warmed to room temperature. Dose calculations were based on average animal weight across a sampling of animals on study. 0.1 ml dose volume was drawn into a 1 cc single dose syringe. Taxol dosages were drawn into a 1 cc single dose syringe q.s. to 0.1 ml. The animals were observed, and clinical signs noted, approximately 4 hours post dose, then monitored for duration of life against no treatment controls. Average group weights were monitored weekly for the duration of the study. Results of this experiment are depicted in FIG. 8. At the conclusion of the study, survival rates for PACLIMER groups were between 14% and over 80% higher, on average over 40% higher, than the control group.

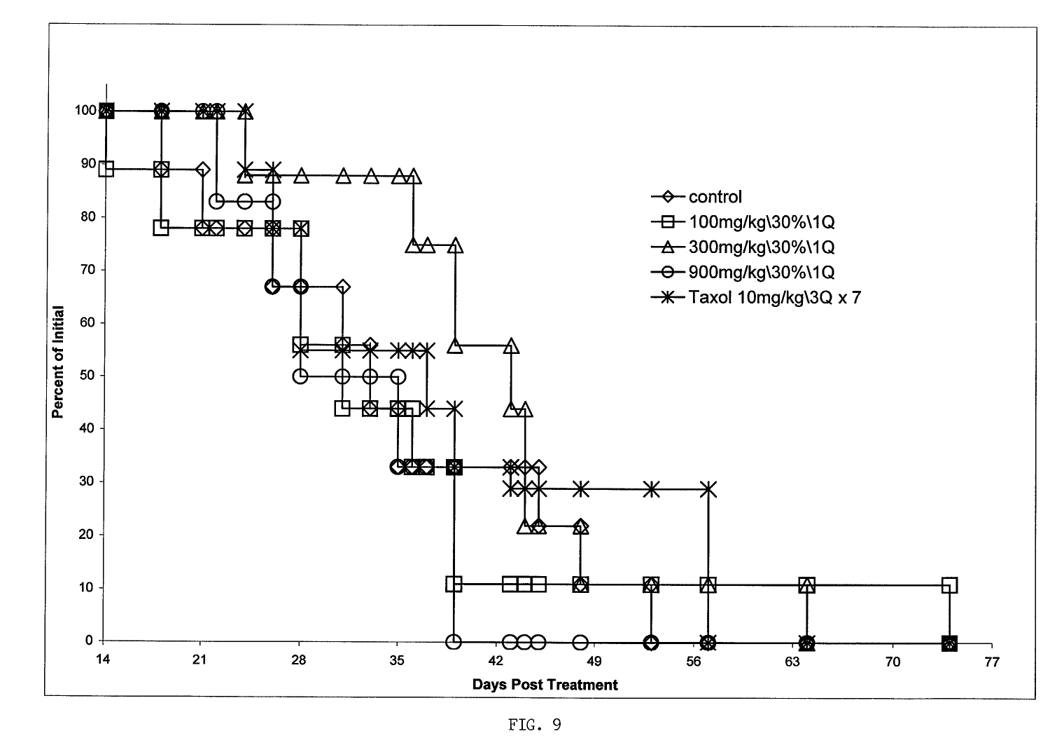

A second experiment was performed using 60 female C57BL/6 mice, 5-6 weeks old at study onset, divided into groups of 10 for control, 100, 300, 900 mg/kg PACLIMER (30%), and 10 mg/kg Taxol 3Q×7 with 10 sentinels to confirm metastases. Under anesthesia, 5.0×10⁵ Lewis Lung murine carcinoma cells per animal were administered through the tail vein in 0.1 ml of RPMI cell culture media. The animals were randomly divided into treatment groups at cell inoculation. At 7 days metastases were confirmed in 2 of 2 sentinel animals, and treatments were carried out. Spray-dried microspheres of PACLIMER (30%) were dosed IV through the tail vein suspended in saline/tween vehicle. Dose materials were kept refrigerated until use, then warmed to room temperature. Dose calculations were based on average animal weight of this strain at this age. Appropriate dose volumes were drawn into a 1 cc multi-dose syringe. Each animal was treated with 0.1 ml into the tail vein. The animals were observed, and clinical signs noted, approximately 4 hours post dose, then monitored for duration of life against no treatment controls. Results are illustrated in FIG. 9. Median survival relative to the control group was increased over 30% in the group receiving 300 mg/kg PACLIMER (30%).

### EXAMPLE 17

### In Vitro Plasma Concentrations of Paclitaxel as a Function of Dose

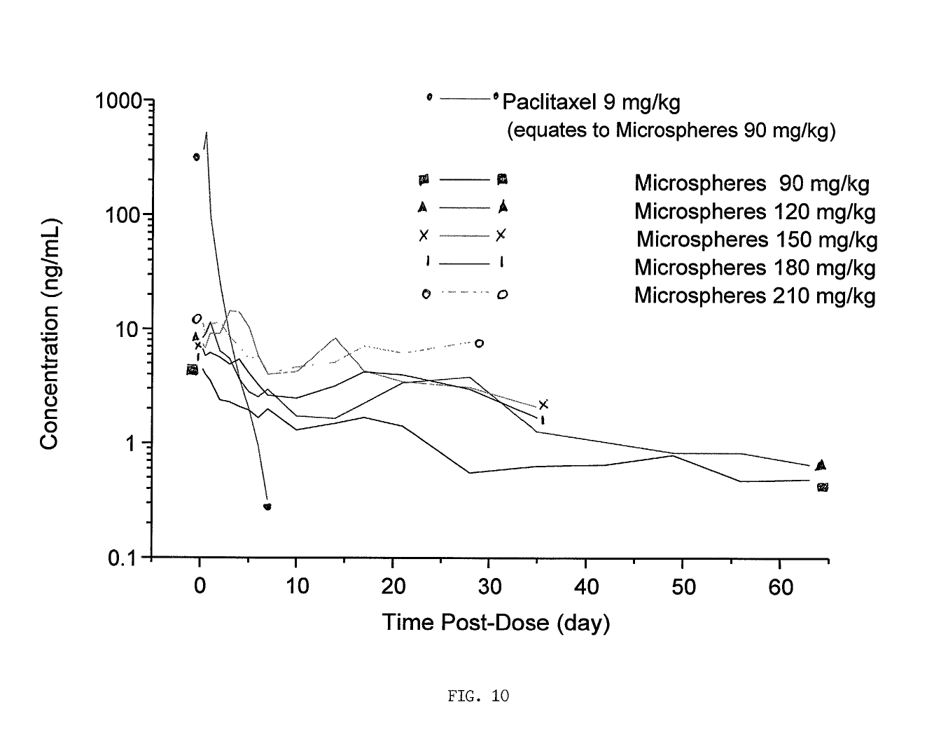

PACLIMER (30%) microspheres were administered intraperitoneally to dogs at doses of 90, 120, 150, 180 and 210 mg/kg; paclitaxel was administered at doses of 9 mg/kg and 12 mg/kg to other dogs. Each dose group comprised one female dog. Plasma samples were taken from each dog at various time points. Samples were analyzed using a LC/MS/MS method. Results are depicted in FIG. 10.

Following administration of 90 and 120 mg/kg doses of PACLIMER (30%) microspheres, plasma samples were collected through 63 days. At doses of 150 and 180 mg/kg, the last sample was obtained at 35 days. The last sampling time for the 210 mg/kg dose was 28 days. The terminal blood sample at each dose level had measurable paclitaxel concentrations. Plasma paclitaxel concentration ranged from 0.6 to 16.9 nM over all time points for all doses.

Following intraperitoneal administration of paclitaxel (9 mg/kg), maximum plasma paclitaxel concentration was reached at 0.5 days and it declined in a biphasic manner and could not be detected beyond 7 days post dose. The animal which received a 12 mg/kg dose of paclitaxel was euthanized before the completion of the study due to toxicity.

### EXAMPLE 18

### Incorporating Paclitaxel into a Phosphorous Linked Polymer

The four methods listed below may be used to prepare microspheres or microparticles of the subject compositions. The subject methods were used to prepare PACLIMER with various loading levels of paclitaxel.

Method I—Spray Drying: 10 g of a phosphorous linked polymer, e.g., P(D,L-APG-EOP), is dissolved in methylene chloride at a concentration of about 10%. After the polymer is completely dissolved, an appropriate amount of paclitaxel powder (e.g., 1.1 g for 10% loading, 4.2 g for 30% loading, 10 g for 50% loading, etc.) is added to the solution and stirred until the powder is completely dissolved. Microspheres are then prepared using a spray-drying technique, e.g., using a Buchi Mini Spray Dryer (Model B-191) at inlet temperature of 35° C., pump rate of 16% (˜10 gm/min) for polymer solution and 800 L/hr for atomizer gas (nitrogen), and aspiration at 50% (˜20 mbar). In most instances, the mean diameter of the resulting microspheres for PACLIMER at various loading levls is less than 20 microns.

Method II—Solvent Evaporation: Microparticles of the subject compositions will be prepared by solvent evaporation. For example, the subject polymer composition and paclitaxel are dissolved in ethyl acetate, the ethyl acetate solution is then emulsified into a 0.5% polyvinylalcohol (PVA) solution presaturated with ethyl acetate at a stirring rate of 600 rpm, followed by the application of a vacuum (e.g., about 15 inches of Hg) to remove the ethyl acetate. For one preferred process, the ethyl acetate concentration will be reduced to below 10% within 10 minutes. Microparticles will be washed on an appropriate sieve with deionized water and thereafter lyophilized.

Method III—Solvent Dilution: Microspheres may be prepared by a solvent dilution method using an in-line homogenizer. For example, approximately 50 grams of paclitaxel and 450 grams of subject polymer composition were weighed and dissolved in 1 L of ethyl acetate. The non-solvent phase was pre-saturated with ethyl acetate; ethyl acetate (800 ml) was added to 9 liter of 0.5% PVA and homogenized for 1 minute. The paclitaxel-subject polymer composition solution and the PVA-ethyl acetate solution were pumped simultaneously through an in-line homogenizer into a container at rates of 1 and 3 liters/min, respectively. The combined solution was gently stirred with an overhead stirrer. Approximately 90 liters of water was added to the container at a rate of 3 L/min. The solution was then gently stirred for 30 minutes. The microsphere suspension was transferred to a filtering/drying unit containing 150 μm scalping and 25 μm product sieves. The resulting microspheres were rinsed with 5 liters of de-ionized water and dried for 3 days under vibration, vacuum and a nitrogen purge. The dried microspheres on the 25 μm sieve were collected into a container.

Method IV—Freeze/pulverize: Microparticles are prepared by evaporating the drug/polymer in solution at 40° C. under a nitrogen purge to obtain viscous mass which is subsequently cooled to −40° C., lyophilized, e.g., for 48 hours, and pulverized to a desired size for the microparticles.

### EXAMPLE 19

### Large-Scale Preparation of D,L-PL(PG)EOP

A 100 g portion of propylene glycol was added to a 3000 ml 3-necked round bottom flask equipped with a gas joint, a stirrer bearing/shaft/paddle assembly, and a Teflon-coated thermocouple. The reaction apparatus was placed in a preheated oil bath at 130° C. and purged with nitrogen for one minute. A 2000 g portion of D,L-lactide was added using a powder addition funnel over a period of 45 minutes. The reaction apparatus was then immersed in the oil so that the oil level was at the bottom of the ground glass joints. The mixture was stirred until all of the solid monomer had melted and the internal temperature had reached approximately 125° C. At this time, a volume of solution of stannous octoate in chloroform equivalent to approximately 400 ppm (117 ppm Sn) was added to the melt using a syringe. The mixture was allowed to stir for approximately 3-16 hours Then oil bath set point was decreased to approximately 125° C. and any residual unreacted monomer removed using vacuum over approximately 1 hour.

A 2500 ml portion of chloroform was used to dissolve and transfer the prepolymer to a pre-chilled, 20-liter jacketed reactor, which contained 2.5 equivalents (based on propylene glycol) of triethylamine and 0.5 equivalents of DMAP dissolved in 3600 ml of chloroform. The reactor was equipped with a stirrer bearing/shaft/turbine assembly, a gas joint, a tubing adapter, and a Teflon-coated thermocouple. With stirring and chilled recirculation on the jacket, the solution was cooled to below −15° C. A solution of 1 equivalent (based on propylene glycol, approximately 215 g) of distilled ethyl dichlorophosphate (EOPCl₂) in 650 ml chloroform was prepared in a 1000 ml 3-necked round bottom flask equipped with a tubing adapter and a gas joint. The EOPCl₂/chloroform solution was added using a piston pump and Teflon tubing over a period of 50 minutes, maintaining the internal temperature at approximately −10° C. Tubing was connected to the gas joints of the flask and reactor to equalize the pressure during the addition. Following the addition, a 50 ml portion of chloroform was added to rinse the flask, feed lines, and pump. The reaction mixture was stirred for 1 hour at low temperature (−8° C. after 1 hour) before the reaction was quenched with 140 ml of anhydrous methanol.

The reactor was then charged with 3 kg of Dowex DR-2030 IER and 3 kg of Dowex M-43 wetted with approximately 6.5 liters of methylene chloride. The polymer/resin mixture was mixed at low temperature for 3-15 hours, after which it was transferred by vacuum to a stainless steel laboratory Nutsche filter. After filtering off the resin, the polymer solution was pulled through the in-line 8 micron cartridge filter into the concentrator (a similar 10-liter jacketed reactor) where the solution was concentrated with the aid of heated recirculating fluid on the jacket. The 20-liter reactor and the resin in Nutsche were washed with 5 liters of methylene chloride, which were transferred to the concentrator after being stirred for 1 hour. An additional 5 liters of methylene chloride were added to the resin in the Nutsche and added to the concentrator when the solution had been reduced to approximately 6 liters.

Concentration of the polymer solution continued until approximately 4-5 liters of a viscous solution remained. A portion of 1500 ml of ethyl acetate was then added to the polymer solution. The mixture was mixed until homogenous and precipitated in approximately 10 liters of petroleum ether. After the precipitation mixture was stirred for approximately 5 minutes, the supernatant liquid was decanted. The polymer was then washed with 5 liters of petroleum ether. After the mixture was stirred for 5 minutes. The liquid was again decanted. The polymer was poured into a Teflon-coated pan and placed in the vacuum oven at NMT 50° C. After drying for 24 hours, the polymer was ground into smaller pieces and dried for additional time in a vacuum oven at ambient temperature.

### REFERENCES

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Patents and Patent Applications

U.S. Pat. Nos. 4,638,045, 5,219,564, 5,099,060, 6,040,330, 6,017,935, 6,002,023, 5,990,325, 5,981,564, 5,977,164, 5,977,163, 5,972,992, 5,922,754, 5,919,815, 5,908,835, 5,912,263, 5,902,822, 5,877,205, 5,854,278, 5,840,929, 5,821,363, 5,817,840, 5,808,888, 5,795,909, 5,780,653, 5,773,464, 5,773,461, 5,767,297, 5,767,296, 5,760,072, 5,756,776, 5,750,691, 5,739,359, 5,728,687, 5,719,177, 5,693,666, 5,688,977, 5,686,623, 5,670,536, 5,614,645, 5,608,087, 5,597,931, 5,908,835, 6,005,120, 5,424,073, and 5,547,981.

Publications and Other References

Ertel et al., (1995) J. Biomedical Materials Res. 29:1337-1348

Choueka et al., (1996) J. Biomed. Materials Res., 31:35-41

Langer et al., (1983) Rev. Macro. Chem. Phys. C23(1):61

Leong et al. (1986) Biomaterials, 7:364

Sato et al., (1996) Bio. Pharm. Bull. 19:1596-601

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition comprising: bicompatible microparticles comprising: (a) a biocompatible polymer having one or more monomeric units represented by the following formula: 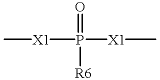
wherein, independently for each occurrence of said monomeric unit:
X1, each independently, represents —O— or —N(R5)—;
R5 represents —H, aryl, alkenyl or alkyl; and
R6 is any non-interfering substituent; and
(b) at least about twenty percent, by weight of said composition, of an antineoplastic taxane.

2. The composition of claim 1, wherein said microparticles are microspheres.

3. The composition of claim 2, wherein said microspheres are mixed with a pharmaceutically acceptable carrier.

4. The composition of claim 1, wherein said polymer is biodegradable.

5. The composition of claim 1, wherein the mean diameter of said microspheres is less than about 250 microns.

6. The composition of claim 1, wherein the mean diameter of said microspheres is less than about 150 microns.

7. The composition of claim 1, wherein said taxane is at least about twenty percent to about sixty percent by weight of said composition.

8. The composition of claim 1, wherein said taxane is at least about thirty percent by weight of said composition.

9. The composition of claim 7, wherein at least about 50 percent of the repeating units of said polymer comprises said monomeric units.

10. The composition of claim 1, wherein each occurrence of X1 for each of said monomeric units represents O.

11. The composition of claim 1, wherein each occurrence of R6 for each of said monomeric units represents H, alkyl, —O-alkyl, —O-cycloalkyl, aryl, —O-aryl, heterocycle or —O-heterocycle.

12. The composition of claim 1, wherein said polymer has one or more monomeric units represented by the following Formula V: 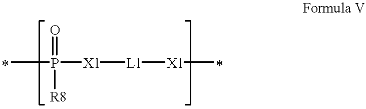
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, alkyl, —O -alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R 11 represents —H, alkyl, aryl, cycloalkyl, cycloalkenyl, heterocycle or polycycle.

13. The composition of claim 12, wherein at least about 25 percent of the repeating units of said polymer comprises said monomeric units.

14. The composition of claim 12, wherein said polymer comprises at least about two of said monomeric units.

15. The composition of claim 12, wherein said polymer comprises at least about five of said monomeric units.

16. The composition of claim 12, wherein each X1 is O.

17. The composition of claim 15, wherein L1 for each of said monomeric units of said polymer represents a divalent branched or straight chain or cyclic aliphatic group or divalent aryl group.

18. The composition of claim 12, wherein L1 for at least one of said units has 2 to about 20 atoms of carbon, oxygen, sulfur and nitrogen, wherein at least 60 percent of said atoms are carbon.

19. The composition of claim 15, wherein L1 represents an alkylene, alkenylene or alkynylene group.

20. The composition of claim 12, wherein L1 comprises a biodegradable polymer selected from the group consisting of polylactide, polyglycolide, polycaprolactone, polycarbonate, polyethylene terephthalate, polyanhydride, polyorthoester, polymers of ethylene glycol and polymers of propylene glycol.

21. The composition of claim 1, wherein said polymer has one or more monomeric units represented by the following Formula VI: 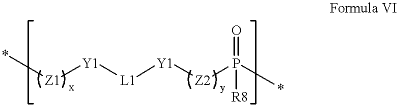
wherein Z1 and Z2, respectively, for each independent occurrence is: 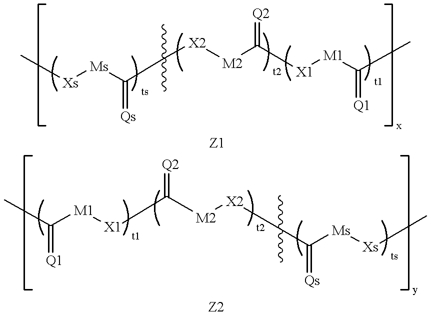
wherein, independently for each occurrence of said monomeric unit:
Q1, Q2. . . Qs, each independently, represent —O— or —N(R7);
X1, X2. . . Xs, each independently, represent —O— or —N(R7);
R7 represents —H, aryl, alkenyl or alkyl;
the sum of t1, t2. . . ts is an integer and equal to at least one or more;
Y1 represents —O—, —S— or —N(R7)—;
x and y are each independently integers from 1 to about 1000 or more;
L1 represents any chemical moiety that does not materially interfere with the biocompatibility of said polymer;
M1, M2. . . Ms each independently, represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

22. The composition of claim 21, wherein said polymer comprises at least about two of said monomeric units.

23. The composition of claim 21, wherein said polymer comprises at least about five of said monomeric units.

24. The composition of claim 21, wherein said monomeric units comprise at least about 95 percent of the repeating units of said polymer.

25. The composition of claim 22, wherein the average molar ratio of (x or y):L1, when ts is equal to one, is from about 10:1 to about 4:1.

26. The composition of claim 21, wherein L1 represents a divalent branched or straight chain or cyclic aliphatic group or divalent aryl group.

27. The composition of claim 24, wherein L1 has 2 to about 20 atoms of carbon, oxygen, sulfur and nitrogen, wherein at least 60 percent of said atoms are carbon.

28. The composition of claim 21, wherein each Q1, Q2. . . Qs and each X1, X2. . . Xs of each of said monomeric units of said polymer is O.

29. The composition of claim 23, wherein each M1, M2. . . Ms of each of said monomeric units of said polymer represents a divalent aliphatic moiety having from 1 to about 7 carbon atoms.

30. The composition of claim 21, wherein the sum of t1, t2. . . ts equals one for each of Z1 and Z2 and Q1 and X1 is O.

31. The composition of claim 23, wherein said monomeric units are represented by the following Formula VIf:

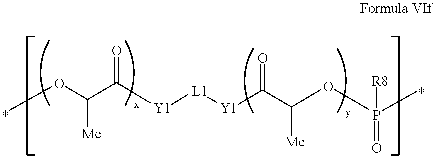

32. The composition of claim 31, wherein each of Y1 represents O.

33. The composition of claim 31, wherein R8 represents —H, alkyl, aryl, —O-alkyl or —O-aryl.

34. The composition of claim 33, wherein said monomeric units comprise at least about 80 percent of said polymer.

35. The composition of claim 31, wherein the chiral carbon for each subunit 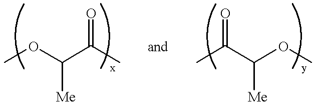
has the D configuration.

36. The composition of claim 31, wherein the chiral carbon for each subunit 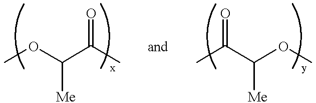
has the L configuration.

37. The composition of claim 22, wherein each of Z1 and Z2 are represented by: 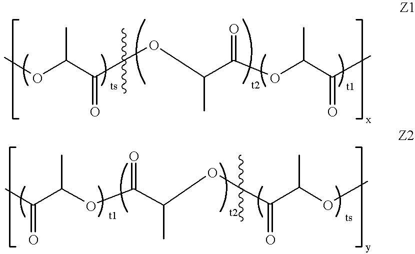
wherein the configuration of the chiral carbon for each ts may be D or L.

38. The composition of claim 21, wherein each of Z1 and Z2 is represented by: 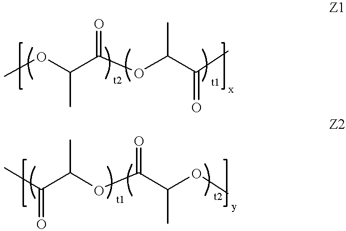
wherein the configuration of the chiral carbons independently for each unit x for Z1 and unit y for Z2 is either D for t1 and L for t2, or L for t1 and D for t2.

39. The composition of claim 38, wherein each of Y1 is O and L1 is —CH(CH₃)CH₂—.

40. The composition of claim 39, wherein said monomeric units comprise at least about 95 percent of said polymer.

41. The composition of claim 1, wherein said polymer has one or more monomeric units represented by the following Formula VII: 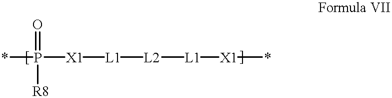
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10, preferably 0-6; and
R11 represents —H, alkyl, aryl, cycloalkyl, cycloalkenyl, heterocycle or polycycle; and
L2 represents a divalent, branched or straight chain aliphatic group, a divalent cycloaliphatic group, a phenylene group, or a group of the formula:

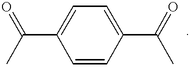

42. The composition of claim 41, wherein each of L1 is —CH₂—.

43. The composition of claim 41, wherein each X1 of each of said units is O.

44. The composition of claim 1, wherein said polymer has one or more monomeric units represented by the following Formula VIII: 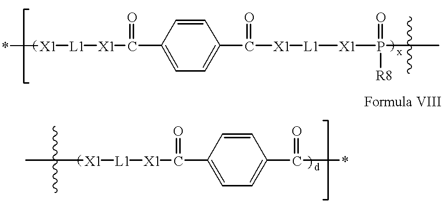
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10, preferably 0-6;
R11 represents —H, alkyl, aryl, cycloalkyl, cycloalkenyl, heterocycle or polycycle; and
d is equal to one or more and x is equal to or greater than one.

45. The composition of claim 44, wherein each L1 independently represents an alkylene group, a cycloaliphatic group, a phenylene group or a divalent group of the formula: 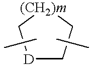
wherein D is O, N or S and m is an integer from 0 to 3.

46. The composition of claims 1, 12, 21, 41 or 44, wherein said taxane is paclitaxel or docetaxel.

47. The composition of claims 1, 12, 21, 41 or 44, wherein said taxane has a structure of Formula III: 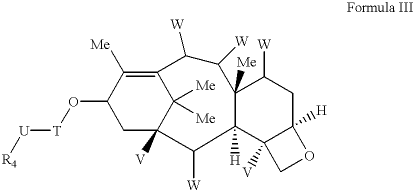
wherein, independently for each occurrence:
V, each independently, represents H, hydroxy, lower alkoxy, or a small ester (e.g., less than 4 carbons);
W, each independently, represents H, hydroxy, carbonyl, amino, alkoxy, sulfhydryl, alkylthio, ester, acylamino, carbamate, sulfonate, carbonate, or sulfoxide;
T represents —C(═O)—, —C(═S)—, —SO₂—, or —SO—;
U is absent or represents NH, S, or O; and
R4 represents a substituted aralkyl.

48. A kit containing a drug delivery system, comprising microparticles and instructions for using said microparticles, wherein said microparticles comprise a therapeutically effective amount of any one of the compositions claimed above.

49. A method for treating or preventing a disease or condition, comprising administering to a patient a therapeutically effective amount of any one of the compositions claimed above.

50. A method for treating or preventing a disease or condition, comprising administering intravascularly to a patient microparticles of a composition comprising (a) a biocompatible polymer, and (b) at least about ten percent by weight of said composition of an antineoplastic taxane, whereupon therapeutically effective levels of said taxane are sustained in the plasma of said patient for a period of at least about seven days.

51. A method for treating or preventing a disease or condition, comprising administering intravascularly to a patient microparticles of a composition comprising (a) a biocompatible polymer, and (b) at least about ten percent by weight of said composition of an antineoplastic taxane, whereupon a therapeutically effective amount of said taxane is released from said composition over a period of at least about seven days.

52. A method for treating or preventing a disease or condition, comprising administering intramuscularly or subcutaneously to a subject microparticles of a composition comprising (a) a biocompatible polymer, and (b) at least about five percent by weight of said composition of an antineoplastic taxane, wherein therapeutically effective levels of said taxane are sustained in the plasma of said patient for a period of at least about ten days after said adminstration.

53. A method for treating or preventing a disease or condition, comprising administering intramuscularly or subcutaneously to a subject microparticles of a composition comprising (a) a biocompatible polymer, and (b) at least about five percent by weight of said composition of an antineoplastic taxane, whereupon a therapeutically effective amount of said taxane is released from said composition over a period of at least about ten days.

54. The method of claim 50, wherein the mean diameter of said microspheres is less than about 20 microns.

55. The method of claim 51, wherein said intravascular adminsitration is intraveous administration.

56. The method of claim 50, wherein said disease or condition is unwanted cell proliferation.

57. The method of claim 51, wherein said disease or condition is an inflammatory disorder.

58. The method of claim 53, wherein said polymer is biodegradable.

59. The method of claim 53, wherein said taxane is at least about ten percent by weight of said composition.

60. The method of claim 5 1, wherein said taxane is at least about twenty percent to about sixty percent by weight of said composition.

61. The method of claim 52, wherein said taxane is at least about thirty percent by weight of said composition.

62. The method of claim 50, wherein said period is at least about fourteen days.

63. The method of claim 52, wherein said period is at least about twenty-one days.

64. The method of claim 52, wherein said period is at least about sixty days.

65. The method of claim 53, wherein said period is at least about ninety days.

66. The method of claims 50, 51, 52 or 53, wherein said polymer is a polymer having phosphorous-based linkages.

67. The method of claim 50, wherein said method increases the median survival rate from said disease or condition by at least about 10 percent, as compared with the median survival rate obtained by administration of a composition comprising the same effective dosage of said taxane not incorporated in said composition.

68. The method of claim 67, wherein said median survival rate obtained by administration of a composition comprising the same effective dosage of said taxane without said polymer is determined by intravenous administration of 135 to 175 mg/m² of said taxane formulated in a pharmaceutically acceptable carrier over about 3 or about 24 hours every three weeks.

69. The method of claim 60, wherein said composition increases the median survival rate for a five year period from said disease or condition by at least about 25 percent, as compared with the median survival rate obtained by administration of a composition comprising the same effective dosage of said taxane without said polymer.

70. The method of claim 69, wherein said taxane is paclitaxel and said taxane without said polymer is formulated in 50 percent CREMOPHOR EL and 50 percent dehydrated alcohol.

71. The method of claim 52, wherein said composition increases the median survival rate for a three year period from said unwanted cell proliferation by at least about 50 percent, as compared with the median survival rate obtained by administration of a composition comprising the same effective dosage of said taxane without said polymer.

72. The method of claim 53, wherein said composition is at least about 75 percent more effective in treating said disease or condition than administration of a composition comprising the same effective dosage of said taxane formulated in a pharmaceutically acceptable carrier.

73. The method of claim 53, wherein said composition reduces the number of hypersensitivity reactions obtained by administration of said composition by at least about 10 percent, as compared with the number of hypersensitivity reactions obtained by administration of a composition comprising the same effective dosage of said taxane formulated in a pharmaceutically acceptable carrier without said polymer and without premedication.

74. The method of claim 73, wherein said number of hypersensitivity reactions obtained by administration of a composition comprising the same effective dosage of said taxane without said polymer is determined by intravenous administration of 135 to 175 mg/m² of said taxane over about 3 or about 24 hours every three weeks.

75. The method of claim 60, wherein said composition reduces the number of significant hypersensitivity reactions obtained by administration of said composition by at least about 25 percent, as compared with the number of hypersensitivity reactions obtained by administration of a composition comprising the same effective dosage of said taxane without said polymer.

76. The method of claim 50, wherein said composition reduces the number of hypersensitivity reactions obtained by administration of said composition by at least about 50 percent, as compared with the number of hypersensitivity reactions obtained by administration of a composition comprising the same effective dosage of said taxane formulated in a pharmaceutically acceptable carrier and without premedication.

77. The method of claim 52, wherein said composition reduces the number of hypersensitivity reactions obtained by administration of said composition by at least about 75 percent, as compared with the number of hypersensitivity reactions obtained by administration of a composition comprising the same effective dosage of said taxane formulated in a pharmaceutically acceptable carrier without said polymer.

78. The method of claim 50, wherein the amount of said taxane released by said composition over about a three week period is at least equal to about 135 mg/m² of said taxane.

79. The method of claim 60, wherein the amount of said taxane released by said composition over about a six week period is at least equal to about 270 mg/m² of said taxane.

80. The method of claim 52, wherein said composition releases over about a three week period an amount of said taxane that is equal to or greater than the amount of said taxane administered formulated in a pharmaceutically acceptable carrier and without said polymer for treatment of said disease or condition for a three week period.

81. The method of claim 60, wherein said composition releases over about a six week period an amount of said taxane that is equal to or greater than the amount of said taxane formulated in a pharmaceutically acceptable carrier administered without said polymer for treatment of said disease or condition for a six week period.

82. The method of claim 53, wherein said composition releases over about a nine week period an amount of said taxane that is equal to or greater than the amount of said taxane administered without said polymer for treatment of said cell proliferation for a nine week period.
